SEQUENCE LISTING (25 sequences)

SEQ ID NO: 1 - DNA, 89 bp, Homo sapiens
  source 1..89: mol_type = other DNA
cggggttggttgttatctttggttatctagctgtatgagtggtgtggagtcttcataaagctagataaccgaaagtaaaaataacccca

SEQ ID NO: 2 - RNA, 23 nt, Homo sapiens
  source 1..23: mol_type = other RNA
tctttggttatctagctgtatga

SEQ ID NO: 3 - RNA, 22 nt, Homo sapiens
  source 1..22: mol_type = other RNA
ataaagctagataaccgaaagt

SEQ ID NO: 4 - DNA, 85 bp, Homo sapiens
  source 1..85: mol_type = other DNA
aggcctctctctccgtgttcacagcggaccttgatttaaatgtccatacaattaaggcacgcggtgaatgccaagaatggggctg

SEQ ID NO: 5 - RNA, 22 nt, Homo sapiens
  source 1..22: mol_type = other RNA
cgtgttcacagcggaccttgat

SEQ ID NO: 6 - RNA, 20 nt, Homo sapiens
  source 1..20: mol_type = other RNA
taaggcacgcggtgaatgcc

SEQ ID NO: 7 - protein, 382 aa, Homo sapiens
MLTRLFSEPGLLSDVPKFASWGDGEDDEPRSDKGDAPPPPPPAPGPGAPGPARAAKPVPLRGEEGTEATLAEVKEEGELGGEEEEEEEEEEGLDEAEGERPKKRGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGADGAGRFHGSGGPFAMHPYPYPCSRLAGAQCQAAGGLGGGAAHALRTHGYCAAYETLYAAAGGGGASPDYNSSEYEGPLSPPLCLNGNFSLKQDSSPDHEKSYHYSMHYSALPGSRPTGHGLVFGSSAVRGGVHSENLLSYDMHLHHDRGPMYEELNAFFHN

SEQ ID NO: 8 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 9 - protein, 1186 aa, Homo sapiens
MEVDTEEKRHRTRSKGVRVPVEPAIQELFSCPTPGCDGSGHVSGKYARHRSVYGCPLAKKRKTQDKQPQEPAPKRKPFAVKADSSSVDECDDSDGTEDMDEKEEDEGEEYSEDNDEPGDEDEEDEEGDREEEEEIEEEDEDDDEDGEDVEDEEEEEEEEEEEEEEEENEDHQMNCHNTRIMQDTEKDDNNNDEYDNYDELVAKSLLNLGKIAEDAAYRARTESEMNSNTSNSLEDDSDKNENLGRKSELSLDLDSDVVRETVDSLKLLAQGHGVVLSENMNDRNYADSMSQQDSRNMNYVMLGKPMNNGLMEKMVEESDEEVCLSSLECLRNQCFDLARKLSETNPQERNPQQNMNIRQHVRPEEDFPGRTPDRNYSDMLNLMRLEEQLSPRSRVFASCAKEDGCHERDDDTTSVNSDRSEEVFDMTKGNLTLLEKAIALETERAKAMREKMAMEAGRRDNMRSYEDQSPRQLPGEDRKPKSSDSHVKKPYYGKDPSRTEKKESKCPTPGCDGTGHVTGLYPHHRSLSGCPHKDRVPPEILAMHESVLKCPTPGCTGRGHVNSNRNSHRSLSGCPIAAAEKLAKAQEKHQSCDVSKSSQASDRVLRPMCFVKQLEIPQYGYRNNVPTTTPRSNLAKELEKYSKTSFEYNSYDNHTYGKRAIAPKVQTRDISPKGYDDAKRYCKDPSPSSSSTSSYAPSSSSNLSCGGGSSASSTCSKSSFDYTHDMEAAHMAATAILNLSTRCREMPQNLSTKPQDLCATRNPDMEVDENGTLDLSMNKQRPRDSCCPILTPLEPMSPQQQAVMNNRCFQLGEGDCWDLPVDYTKMKPRRIDEDESKDITPEDLDPFQEALEERRYPGEVTIPSPKPKYPQCKESKKDLITLSGCPLADKSIRSMLATSSQELKCPTPGCDGSGHITGNYASHRSLSGCPRAKKSGIRIAQSKEDKEDQEPIRCPVPGCDGQGHITGKYASHRSASGCPLAAKRQKDGYLNGSQFSWKSVKTEGMSCPTPGCDGSGHVSGSFLTHRSLSGCPRATSAMKKAKLSGEQMLTIKQRASNGIENDEEIKQLDEEIKELNESNSQMEADMIKLRTQITTMESNLKTIEEENKVIEQQNESLLHELANLSQSLIHSLANIQLPHMDPINEQNFDAYVTTLTEMYTNQDRYQSPENKALLENIKQAVRGIQV

SEQ ID NO: 10 - DNA, 20 bp, synthetic construct
  source 1..20: mol_type = other DNA
ccctttgagaacacgacaca

SEQ ID NO: 11 - DNA, 20 bp, synthetic construct
  source 1..20: mol_type = other DNA
tctgttagcggtgctgaggt

SEQ ID NO: 12 - DNA, 21 bp, synthetic construct
  source 1..21: mol_type = other DNA
aagtcgccgtcttccgccaag

SEQ ID NO: 13 - DNA, 21 bp, synthetic construct
  source 1..21: mol_type = other DNA
gtccagggacccaatcttcga

SEQ ID NO: 14 - DNA, 18 bp, synthetic construct
  source 1..18: mol_type = other DNA
acgtgcgcaagttgatga

SEQ ID NO: 15 - DNA, 20 bp, synthetic construct
  source 1..20: mol_type = other DNA
tcacgttgaacccagagatg

SEQ ID NO: 16 - DNA, 19 bp, synthetic construct
  source 1..19: mol_type = other DNA
tcgtgtagtggacgaacgg

SEQ ID NO: 17 - DNA, 21 bp, synthetic construct
  source 1..21: mol_type = other DNA
tctcattgcccatatccaggg

SEQ ID NO: 18 - DNA, 20 bp, synthetic construct
  source 1..20: mol_type = other DNA
cttccacatcgacaccaatg

SEQ ID NO: 19 - DNA, 20 bp, synthetic construct
  source 1..20: mol_type = other DNA
tttggctcagacttgacgtg

SEQ ID NO: 20 - DNA, 23 bp, synthetic construct
  source 1..23: mol_type = other DNA
acaattttcctgactccaaggac

SEQ ID NO: 21 - DNA, 21 bp, synthetic construct
  source 1..21: mol_type = other DNA
actgtgacgaagctcagagac

SEQ ID NO: 22 - DNA, 20 bp, synthetic construct
  source 1..20: mol_type = other DNA
gctgtgggagagagaggaga

SEQ ID NO: 23 - DNA, 21 bp, synthetic construct
  source 1..21: mol_type = other DNA
gcaggctgtagtggttagacg

SEQ ID NO: 24 - DNA, 20 bp, synthetic construct
  source 1..20: mol_type = other DNA
ttgaggtcaatgaaggggtc

SEQ ID NO: 25 - DNA, 20 bp, synthetic construct
  source 1..20: mol_type = other DNA
gaaggtgaaggtcggagtca

(19) United States
(12) Patent Application Publication
(10) Pub. No.: US 2024/0352414 A1
(43) Pub. Date: Oct. 24, 2024

(54) THREE-DIMENSIONAL DIRECT NEURONAL REPROGRAMMING TO MODEL ALZHEIMER'S DISEASE IN HUMAN NEURONS

(71) Applicant: Washington University, St. Louis, MO (US)

(72) Inventors: Andrew Yoo, St. Louis, MO (US); Zhao Sun, St. Louis, MO (US)

(21) Appl. No.: 18/633,066

(22) Filed: Apr. 11, 2024

Related U.S. Application Data
(60) Provisional application No. 63/458,567, filed on Apr. 11, 2023.

(51) Int. Cl. C12N 5/0793; C12N 15/113; C12N 15/86; G01N 33/50

(52) U.S. Cl. CPC C12N 5/0619; C12N 15/113; C12N 15/86; G01N 33/5023; G01N 33/5058; C12N 2310/14; C12N 2501/60; C12N 2506/1307; C12N 2513/00

(57) ABSTRACT

Provided herein are methods of making and using reprogrammed neuronal cells and neuronal spheroids from patient-derived somatic cells in 3D-cultures, by expressing at least one neurogenic miRNA and optionally at least one neurogenic transcription factor.

### GOVERNMENTAL RIGHTS

This invention was made with government support in part under grant number RF1AG056296 awarded by National Institutes of Health/National Institute on Aging. The government has certain rights in the invention.

### FIELD

The present disclosure generally relates to methods and compositions for generating reprogrammed cortical neurons and neuronal spheroids from Alzheimer's disease patient-derived fibroblasts and uses thereof in modeling the neuropathology of Alzheimer's disease and for treating Alzheimer's disease.

### REFERENCE TO THE SEQUENCE LISTING

This application contains a Sequence Listing that has been submitted electronically as a computer readable XML format via Patent Center and is hereby incorporated by reference in its entirety pursuant to 37 C.F.R. § 1.52 (c) (5). The XML file, created on Apr. 10, 2024, is named “047563_793638.xml” and is 24,861 bytes in size.

### BACKGROUND OF THE INVENTION

Alzheimer's disease (AD) is a neurodegenerative disorder that primarily affects elderly individuals and is characterized by hallmark neuronal pathologies including extracellular amyloid-β (Aβ) deposition, intracellular tau tangles, and neuronal death. It is the sixth leading cause of death in the US and the most common cause of dementia in aging. With a rapidly growing aging population the number of AD cases is growing fast and projected to rise drastically over the next three decades. More than 5 million people are currently estimated to have AD in the United States alone, and this number is projected to reach 14 million by 2050. Therefore, AD poses a huge economic burden on society placing overwhelming strain on the healthcare system.

Late onset AD (LOAD) accounts for over 95% of all AD cases and is a heterogeneous disease associated with a variety of risk factors, such as age, genetic predisposition, sex, stroke, and other factors. Among these, aging is the most significant risk factor, in which the risk increases exponentially with advanced age over 65. Due to the complex and heterogeneous nature of LOAD, patient-specific modelling using transgenic animals and cellular approaches has been challenging. Therefore, there is an urgent need to develop a patient-based model to study disease pathogenesis, understand its underlying mechanisms and develop personalized treatment options.

Thus, AD remains an unmet medical need underscoring the urgent need for a paradigm shift in AD clinical research.

### SUMMARY OF THE INVENTION

Disclosed herein is a method of generating a reprogrammed cortical neuron or neuronal spheroids from a patient-derived somatic cell. The method may comprise providing the patient-derived somatic cell. The patient may have been diagnosed with or be suspected of having Alzheimer's disease (AD). The method may comprise expressing miR-9/9*, miR-124, and at least one exogenous transcription factor, in the patient-derived somatic cell. The method may comprise culturing the patient-derived somatic cell in a 3D-culture, thereby reprogramming the somatic cell to a reprogrammed cortical neuron or neuronal spheroid. Non-limiting examples of suitable exogenous transcription factors include NEUROD2, ASCL1 or MYT1L. The disclosed method may not require the induction of pluripotency in the somatic cell. Any patient-derived somatic cell may be used in the disclosed method, for example adult human fibroblast of mesodermal origin. The patient may have been diagnosed with or may be suspected of having Late Onset Alzheimer's disease (LOAD). The patient may be at least 60 years or more of age. The reprogrammed cortical neuron or neuronal spheroids obtained by the method may comprise one or more age signatures of the patient-derived somatic cell, for example epigenetic clock, telomere length, and transcriptomic changes. The reprogrammed cortical neuron or neuronal spheroids may exhibit one or more age-related LOAD neuropathology of the patient. The one or more age-related LOAD neuropathology may comprise extracellular Aβ deposition, dystrophic neurites, hyperphosphorylated, K63-ubiquitin-positive tau, seed-competent tau, spontaneous neuronal death, increased levels of Aβ42, active GSK-3B, phosphorylated tau, endosomal abnormalities, oxidative stress, or any combination thereof.

The disclosed miRNA (miR-9/9* and miR-124), or the at least one exogenous transcription factor, or both may be encoded by a nucleic acid sequence contained in a vector. The nucleic acid sequence may comprise a sequence set forth in SEQ ID NO: 1 and SEQ ID NO: 4, or a sequence at least about 80% identical thereto. The nucleic acid sequence may comprise a sequence set forth in any one or more of SEQ ID NO: 7, SEQ ID NO: 8 and SEQ ID NO: 9, or a sequence at least about 80% identical thereto. The vector may further comprise one or more regulatory elements comprising a promoter, an enhancer, a UTR, a termination sequence, an IRES or any combination thereof, operably linked to the nucleic acid sequence encoding the miRNA or at least one exogenous transcription factor. The promoter may be an inducible promoter. The disclosed vector may be a viral vector selected from adenovirus, adeno-associated virus, a retrograde virus, retrovirus, herpesvirus, lentivirus, poxvirus or papilloma virus expression vector.

Disclosed herein is a method of screening for a drug for treatment of Alzheimer's disease (AD). The method may comprise (a) providing a patient-derived somatic cell; (b) expressing miRNA-9/9*, miRNA-124 and at least one exogenous transcription factor in the patient-derived somatic cell; (c) culturing the patient-derived somatic cell in a 3D-culture, thereby reprogramming the somatic cell to a reprogrammed cortical neuron cell or neuronal spheroid; (d) contacting the reprogrammed cortical neuron cell or neuronal spheroids with the drug; and (c) following step (d), analyzing the treated reprogrammed cortical neuron cell or neuronal spheroids for a level of at least one AD-related neuropathology. A reduction in the level of the at least one AD-related neuropathology in the reprogrammed cortical neuron cell or neuronal spheroids in comparison to a control spheroids may be indicative of the effectiveness of the drug for treating Alzheimer's disease. The AD may be a late-onset AD (LOAD). The at least one AD-related neuropathology analyzed for the disclosed method may comprise extracellular Aβ deposition, dystrophic neurites, hyperphosphorylated, K63-ubiquitin-positive tau, seed-competent tau, spontaneous neuronal death, increased levels of Aβ42, active GSK-3B, phosphorylated tau, endosomal abnormalities, oxidative stress, or any combination thereof.

Disclosed herein are reprogrammed cortical neuron cell or neuronal spheroid, generated by a method which may comprise (a) providing a patient-derived somatic cell; (b) exogenously expressing miRNA-9/9*, miRNA-124, and at least one transcription factor in the patient-derived somatic cell; and (c) culturing the patient-derived somatic cell in a 3D-culture, thereby reprogramming the somatic cell to the reprogrammed cortical neuron cell or neuronal spheroid. The reprogrammed cortical neuron or neuronal spheroids may exhibit one or more age-related phenotype(s) of the patient-derived somatic cell. The one or more age-related phenotype(s) exhibited by the reprogrammed cells may be epigenetic clock, telomere length, and at least one age-related transcriptomic change.

Other objects and features will be in part apparent and in part pointed out hereinafter.

### REFERENCE TO COLOR FIGURES

The patent or application file contains at least one drawing executed in color. Copies of this patent or patent application publication with color drawing(s) will be provided by the Office upon request and payment of the necessary fee.

### BRIEF DESCRIPTION OF THE FIGURES

Those of skill in the art will understand that the drawings, described below, are for illustrative purposes only. The drawings are not intended to limit the scope of the present teachings in any way.

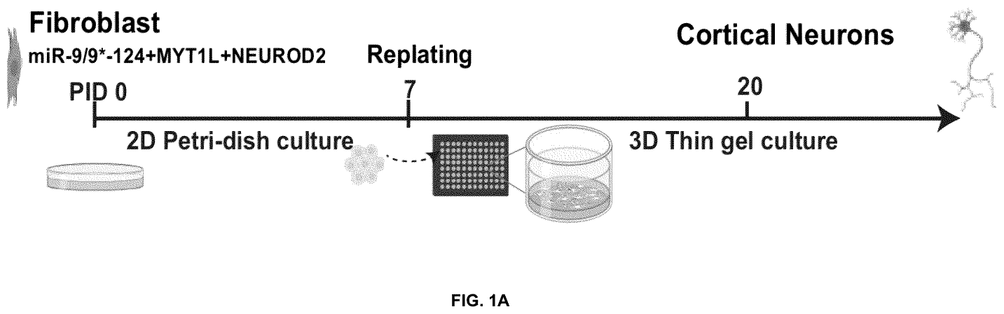

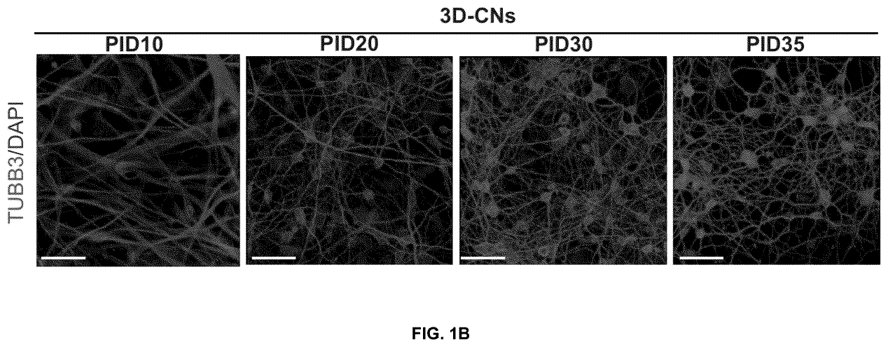

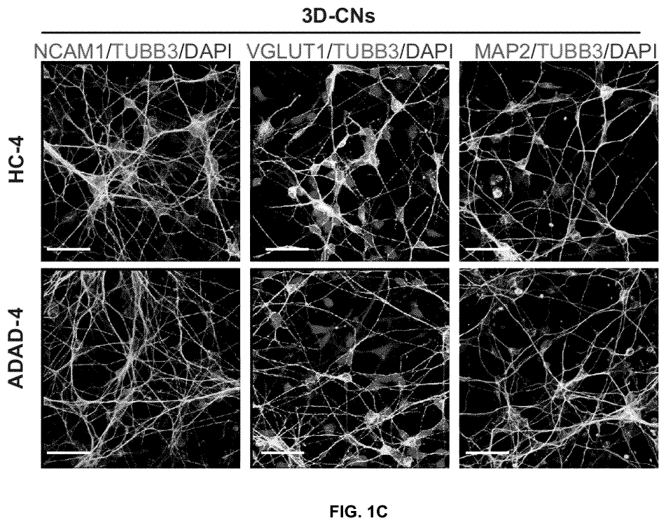

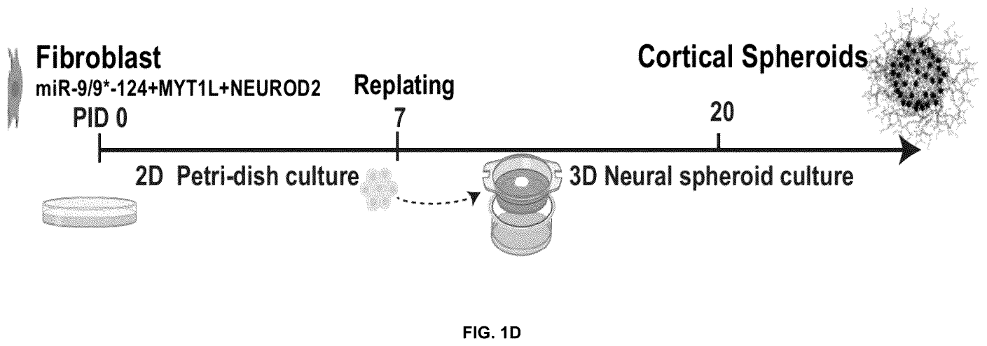

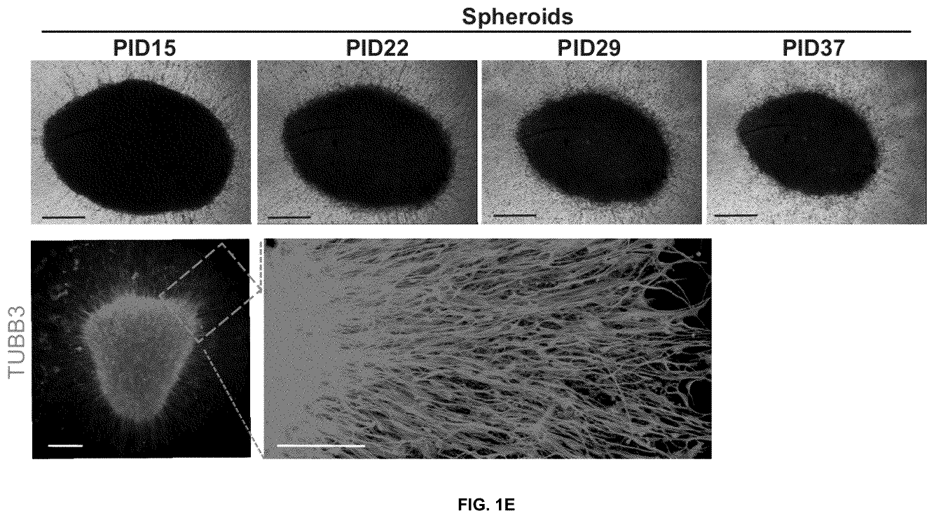

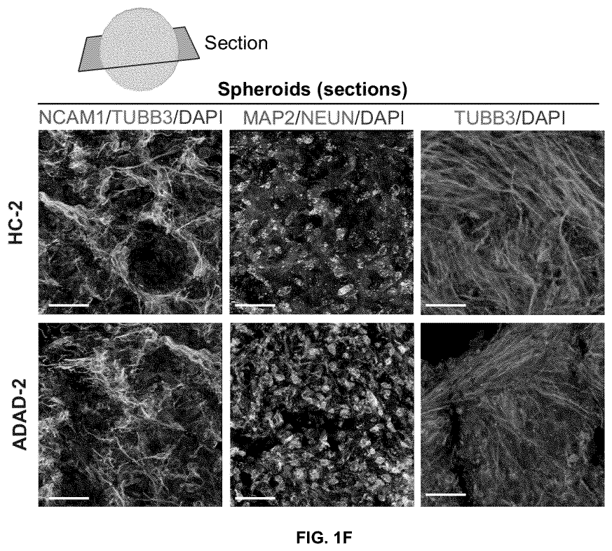

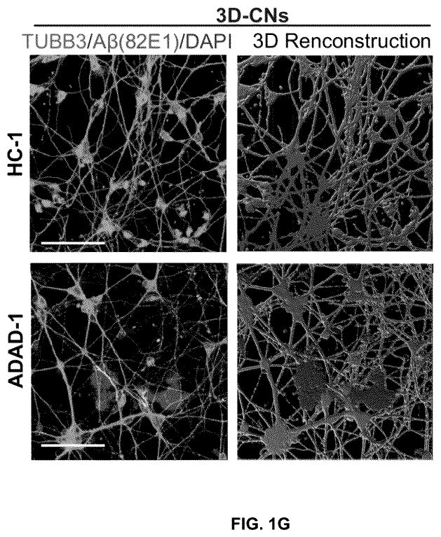

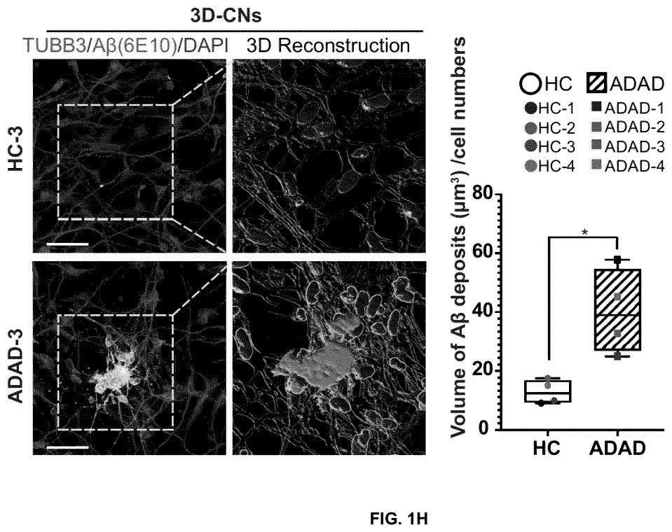

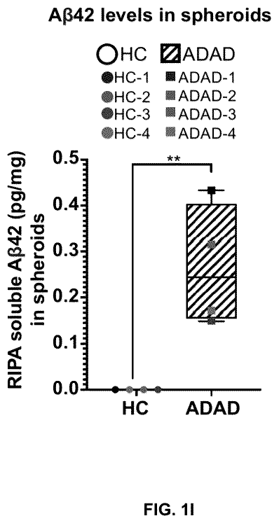

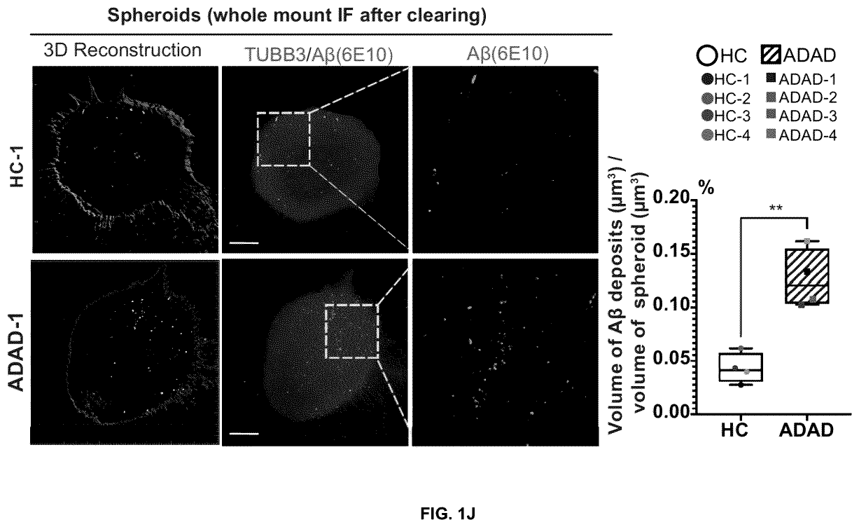

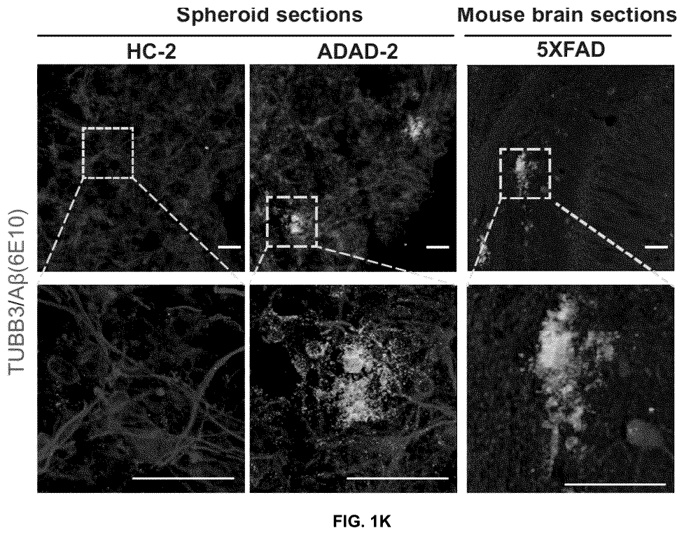

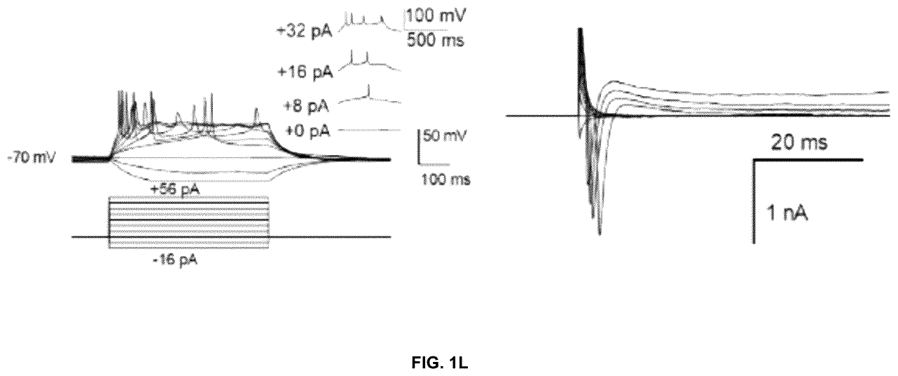

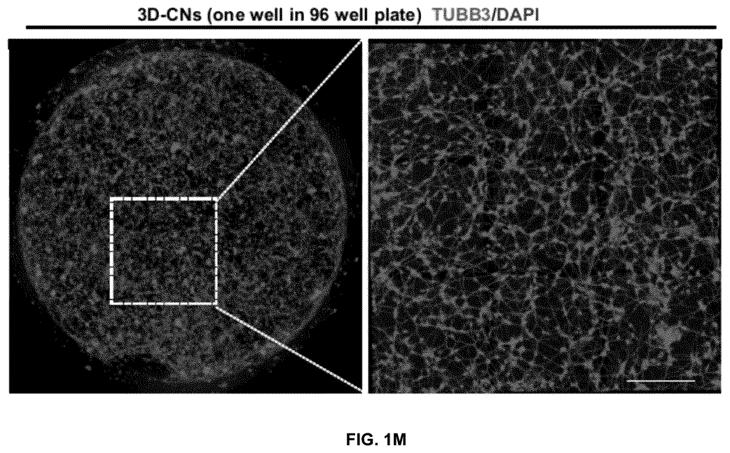

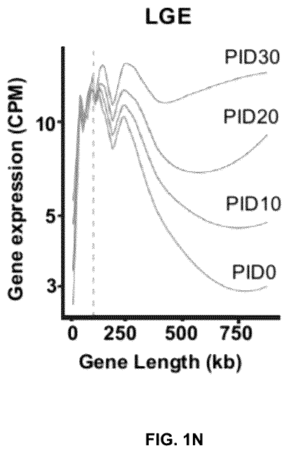

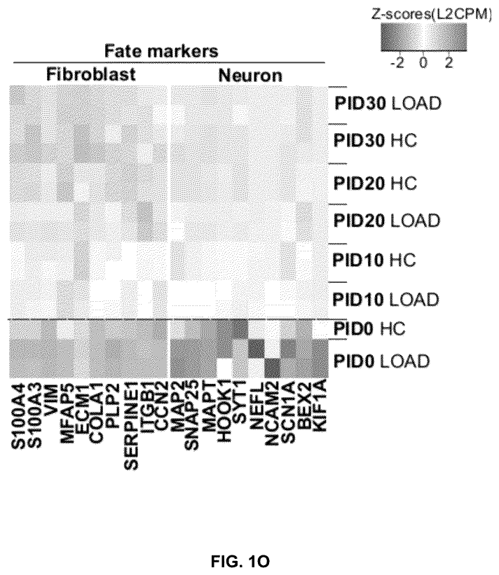

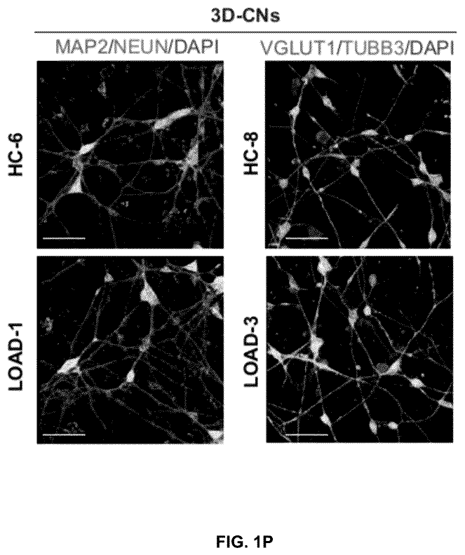

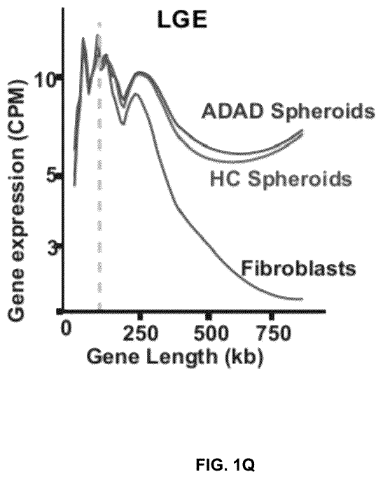

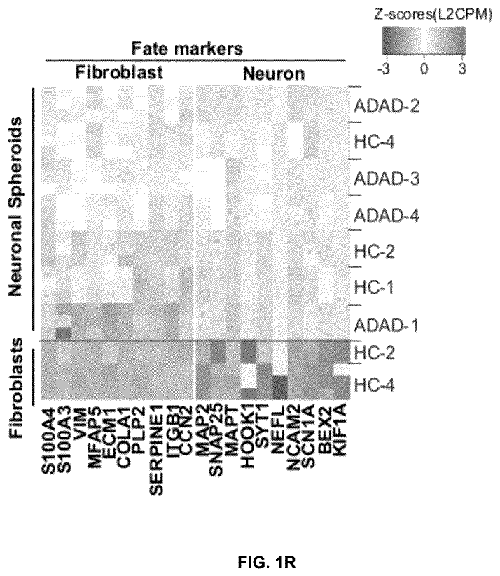

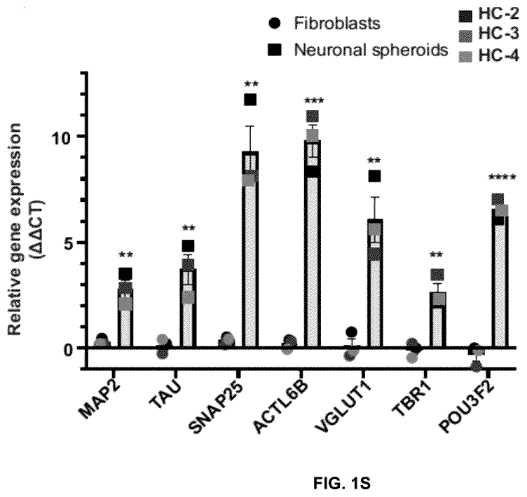

FIGS. 1A-FIG. 1K are a series of illustrations, images, and graphs depicting the direct reprogramming of AD patient fibroblasts into cortical neurons in 3D culture (3D-CNs) and neuronal spheroids and show that the 3D-CNs and spheroids showed higher amount of Aβ deposits. FIG. 1A is a schematic diagram of 3D-direct reprogramming of patient fibroblasts to cortical neurons in thin gel culture (PID: post-induction day). FIG. 1B shows representative immunofluorescence images with TUBB3 staining illustrating morphological changes during neuronal reprogramming in thin gel. Scale bar: 50 μm. FIG. 1C provides representative images of 3D-CNs stained with neuronal markers NCAM1, VGLUT1 and MAP2 at PID30 in thin gel. Scale bar: 50 μm. FIG. 1D is a schematic diagram of direct reprogramming of human fibroblasts to neuronal spheroids per transwell. FIG. 1E Top: Representative phase contrast images of neuronal spheroids at different PIDs of reprogramming. Scale bar: 500 μm. Bottom: A representative image of neuronal spheroids at PID28 stained with TUBB3. Higher magnification view of the boxed region depicts neurite extensions radiating out from the core of the spheroid. Scale bar: 250 μm.

FIG. 1F shows immunostaining in the sections of spheroids for neuronal markers NCAM1, MAP2 and NEUN and morphological marker TUBB3. Scale bar: 50 μm. FIG. 1G shows examples of extracellular Aβ deposits detected by 82E1 Aβ antibody in thin gel at PID30. Scale bar: 50 μm.

FIG. 1H provides representative immunofluorescence images (left) and enlarged 3D reconstruction images (right) showing extracellular Aβ deposition (6E10) in thin gel at PID30. Quantification: total volume (μm³) of Aβ deposition measured using Imaris and was normalized to the total number of cells. Scale bar: 50 μm. FIG. 1I shows Aβ42 levels measured by an electrochemiluminescence assay in ADAD and HC spheroids (PID28). FIG. 1J provides representative images of whole mount immunostaining of Aβ followed by clearing of ADAD spheroids and sex- and age-matched HC spheroids at PID28. 3D reconstruction was generated by Imaris and was used for quantification. Aβ deposition was quantified by measuring the total volume (μm³) of Aβ deposits labeled by 6E10 normalized to the volume (μm³) of spheroids labeled by TUBB3. Scale bar: 500 μm. FIG. 1K provides representative images of spheroids sections (PID28) and 5XFAD mouse brain cortex showing the extracellular Aβ deposition. Scale bar: 50 μm. For quantification in FIGS. H, I, and J, n=4 independent ADAD patients and 4 HC individuals. * p<0.05 and ** p<0.01 were calculated by unpaired t-test.

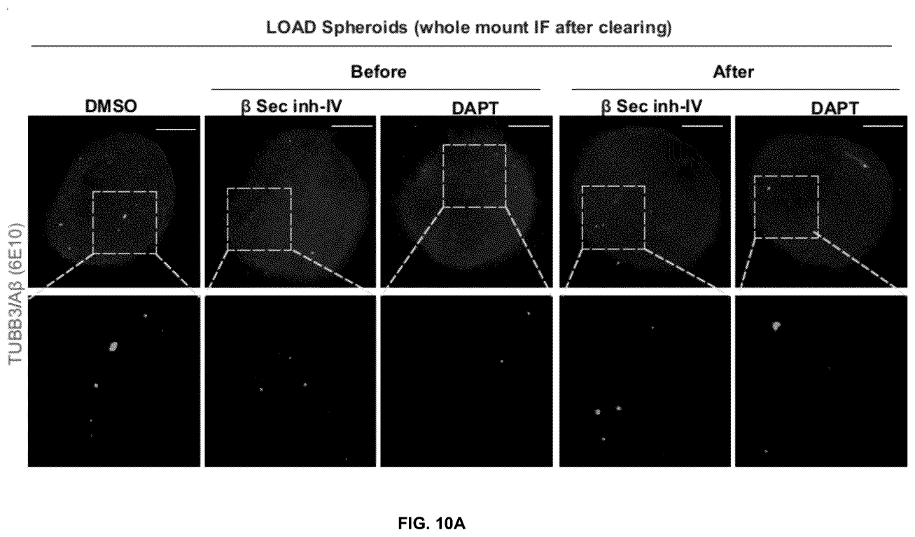

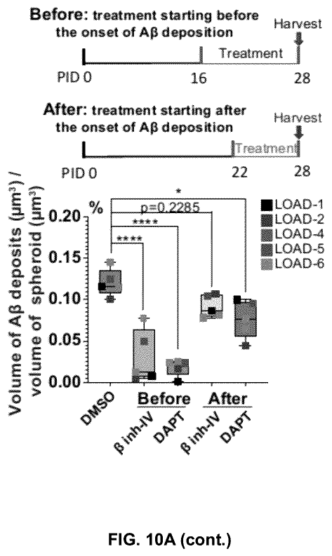

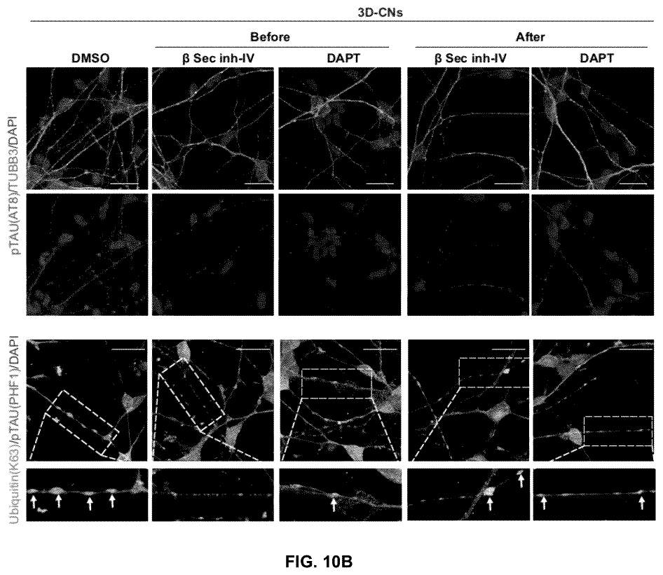

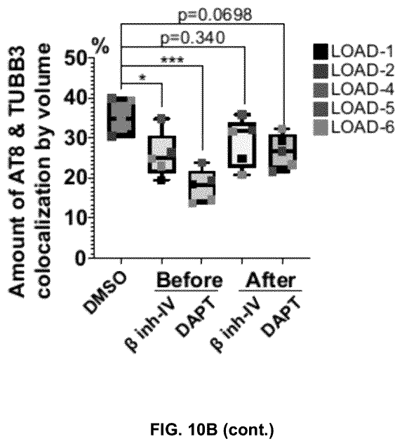

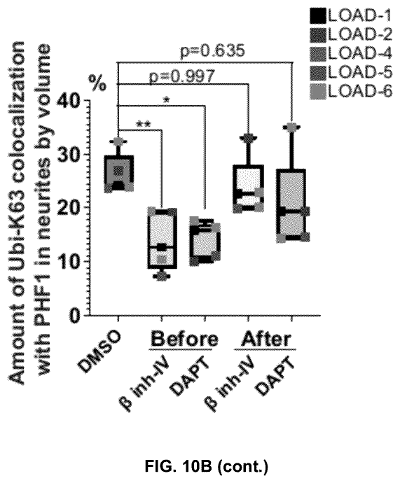

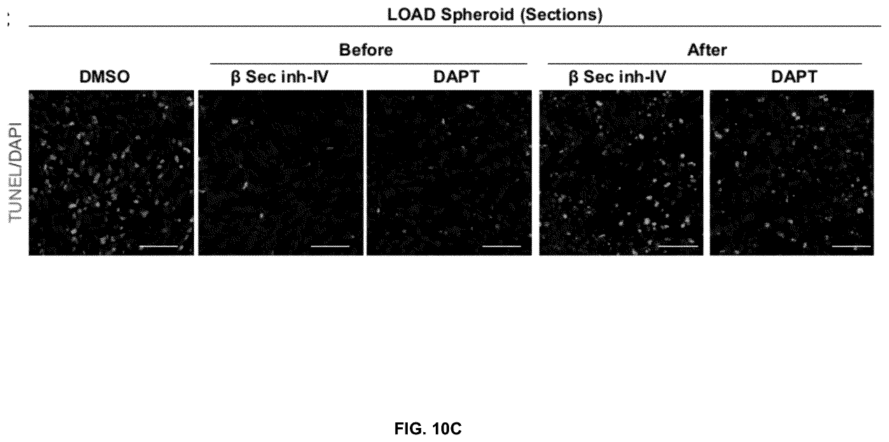

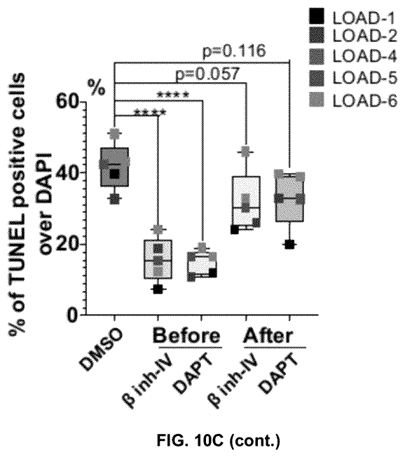

FIGS. 1L-1S show direct reprogramming of patient fibroblasts into 3D-CNs and spheroids FIG. 1L Left: Current-clamp whole cell recording from converted neurons (PID30) in 2D culture environment co-cultured with human astrocytes showing multiple action potentials (APs). Right: Voltage-clamp whole cell recording revealed the inward and outward currents. FIG. 1M Left: Representative images of a thin gel culture (PID28) in one whole well of a 96-well plate. TUBB3 antibody was used to show the neuronal morphology. Right: A magnified image showing the boxed region on the left. Scale bar: 250 μm. FIG. 1N is a LONGO plot depicting increased long gene expression (LGE) during neuronal reprogramming in thin gel. FIG. 10 is a heatmap showing downregulation of fibroblast markers and upregulation of neuronal markers during neuronal reprogramming in thin gel. Replicates from an AD line and HC line were used for analysis. FIG. 1P shows immunostaining for MAP2, NEUN, VGLUT1 and TUBB3 in the thin gel culture of PID30 3D-CNs derived from LOAD patient fibroblasts. Scale bar: 50 μm. FIG. 1Q shows expression of long genes in PID28 ADAD spheroid, PID28 HC spheroid, and their starting fibroblasts. FIG. 1R is a heatmap shows downregulation of fibroblast marker and upregulation of neuronal markers in PID28 neuronal spheroids compared to their starting fibroblasts. Spheroids derived from 4 independent ADAD patients and 3 HC individuals, and fibroblasts from 2 HC individuals were used for analysis. qPCR analysis. FIG. 1S is a bar graph showing mRNA expression of different neuronal markers in neuronal spheroids compared to their starting fibroblasts. n=3 HC individuals. Mean±SEM. ** p<0.01, *** p<0.001 and **** p<0.0001 by multiple unpaired t-tests.

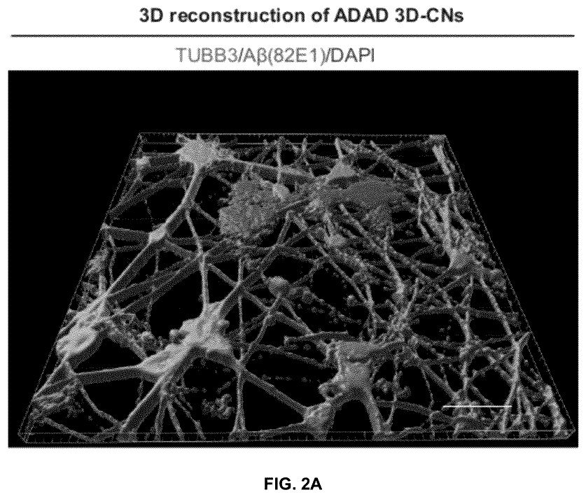

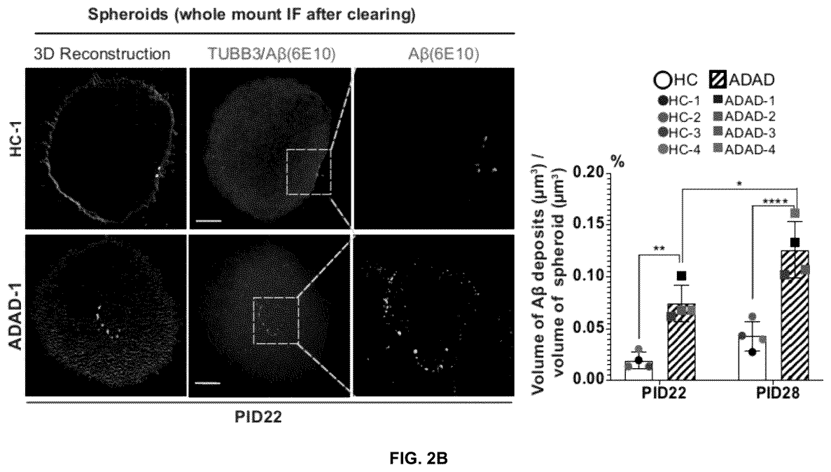

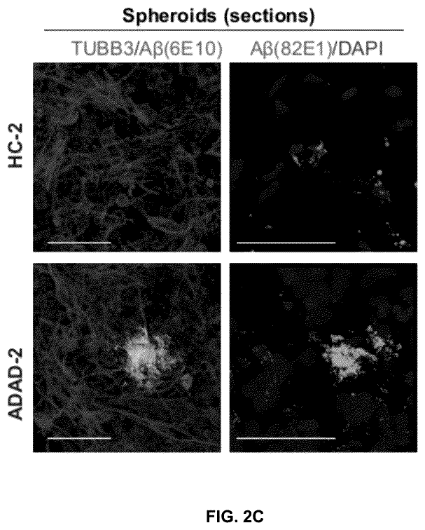

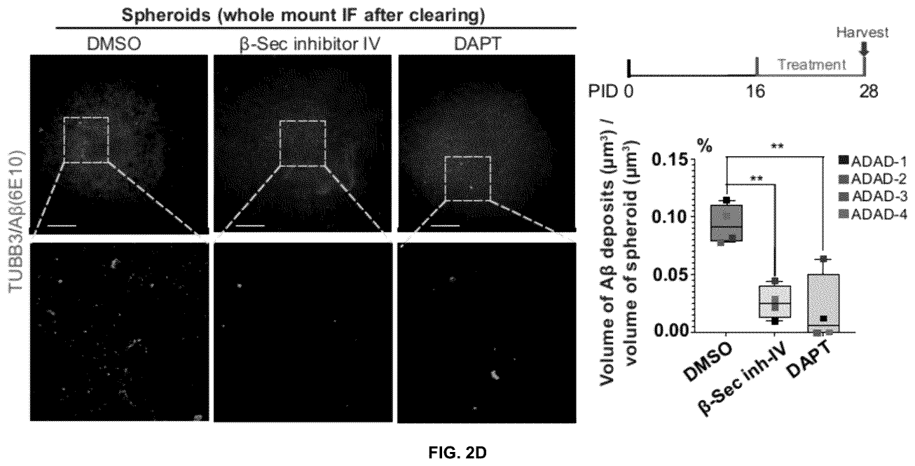

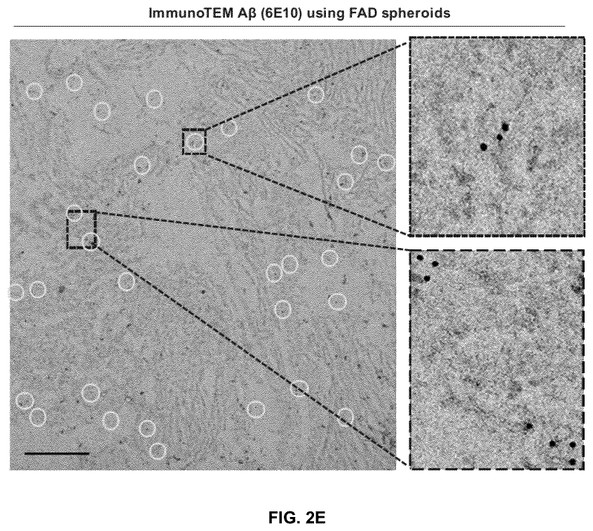

FIGS. 2A-2E provide methods for detection of Aβ deposits in ADAD 3D-CNs and spheroid. FIG. 2A shows a tilted view of a 3D reconstruction image of ADAD 3D-CNs in FIG. 1G depicting the extracellular deposits labeled by 82E1 Aβ antibody (red). Scale bar: 50 μm. FIG. 2B is representative images of whole mount immunostaining of Aβ followed by clearing of ADAD spheroids and sex- and age-matched HC spheroids at PID22. Scale bar: 500 μm. Representative images for Aβ deposition at PID28 can be found in FIG. 1J. p-values were calculated by two-way ANOVA with Šídák's multiple comparisons test. The adjusted p-value for comparison between PID22 HC and PID28 HC was not significant (p=0.1855). * adjusted p<0.05, ** adjusted p<0.01, and **** adjusted p<0.0001. FIG. 2C provides representative immunofluorescence images of the sections of spheroids using two Aβ antibodies (left: 6E10; right: 82E1) showing the extracellular Aβ deposition. Scale bar: 50 μm. FIG. 2D is representative immunofluorescence images and quantification of ADAD spheroids treated with 5 μM β-secretase inhibitor IV or 40 μM DAPT starting at PID16 and immunostained for Aβ and TUBB3 at PID28. Adjusted p-values were calculated by one-way ANOVA with Dunnett's multiple comparisons test. ** p<0.01. Scale bar: 500 μm. FIG. 2E provides transmission EM images from ADAD spheroids showing extracellular signals of immuno-gold-labeled Aβ antibody (6E10). Yellow circles highlight the nanogold particles conjugated to Aβ antibody. Scale bar: 1 μm. For quantification in B and D, n=4 independent ADAD patients and 4 HC individuals.

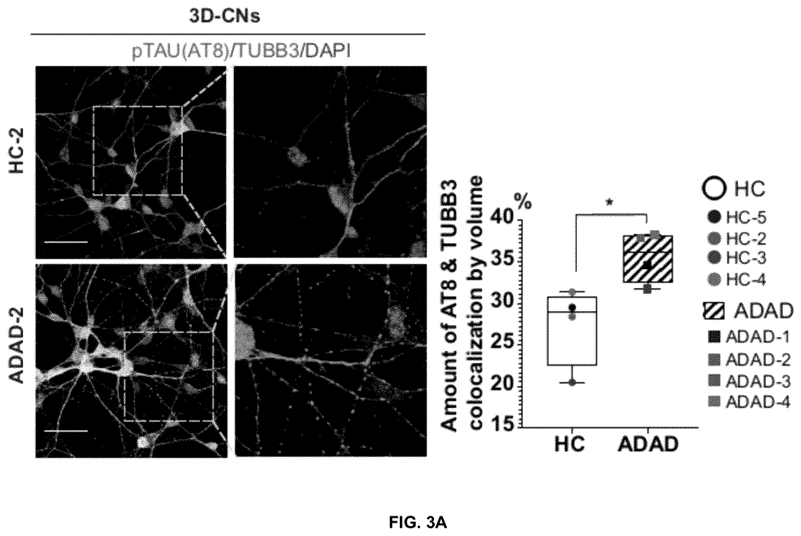

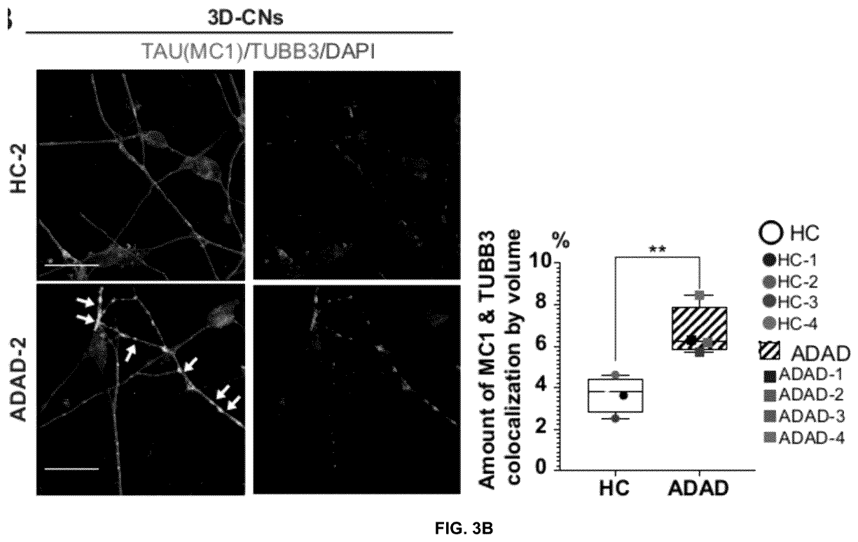

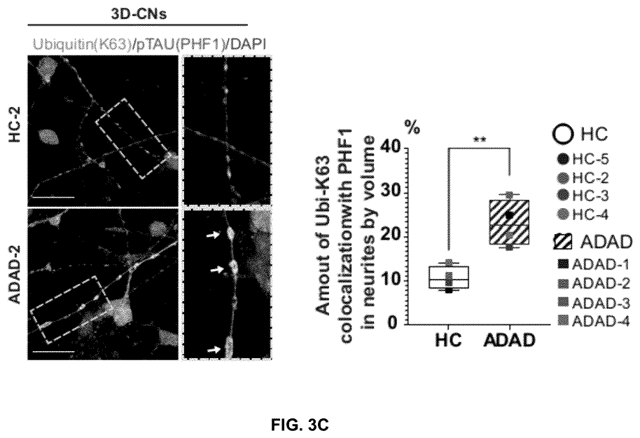

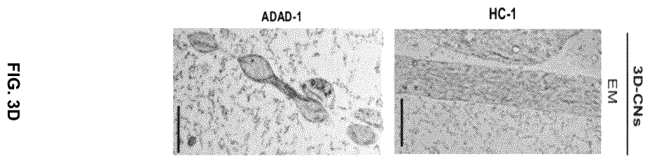

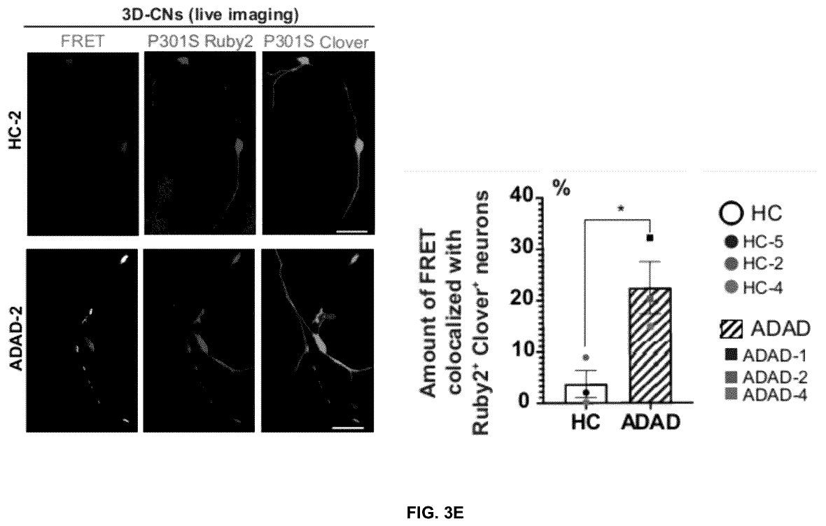

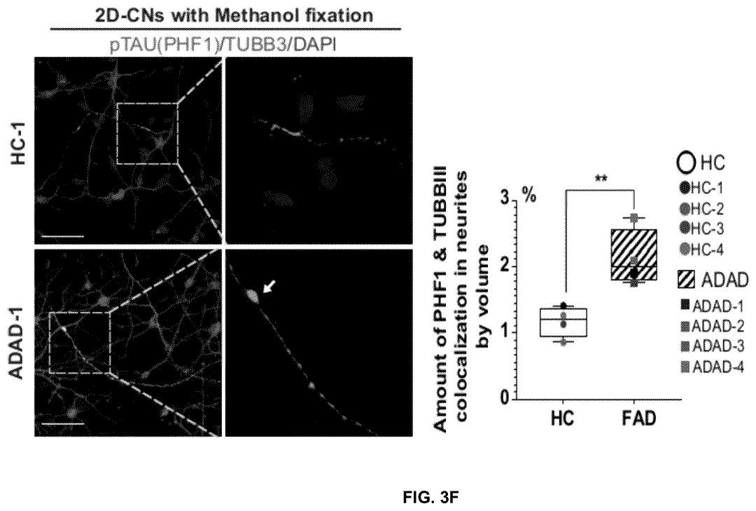

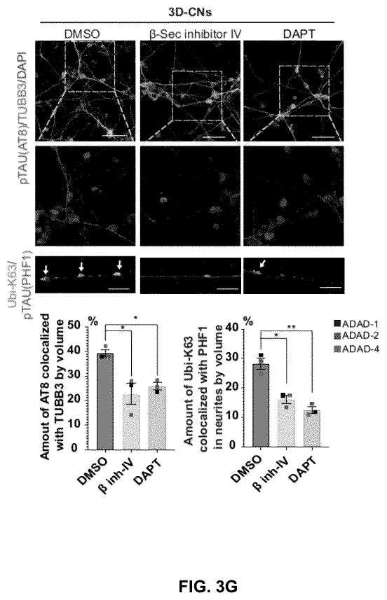

FIGS. 3A-3G provide data from experiments conducted to identify tauopathy in FAD 3D-CNs. FIG. 3A shows immunofluorescence staining of p-tau (AT8 antibody) in ADAD and HC 3D-CNs (PID30). Scale bar: 50 μm. For quantification in n=4 independent ADAD patients and 4 HC individuals. FIG. 3B shows misfolded tau detected by immunostaining with MC1 tau antibody in ADAD and HC 3D-CNs at PID30. Arrows highlight the tau islands that are positive for MC1 antibody. Scale bar: 25 μm. For quantification, n=4 independent ADAD patients and 4 HC individuals. FIG. 3C shows immunofluorescence images of pathogenic tau by co-staining of K63-linked ubiquitin (green) and p-tau (PHF1) (red) in ADAD and HC 3D-CNs. Arrows point to the enlarged beaded neurite bulges with PHF1 signal colocalized with K63-linked Ubiquitin. Scale bar: 25 μm. For quantification, n=4 independent ADAD patients and 4 HC individuals. FIG. 3D is representative transmission EM images of healthy neurites (top) and dystrophic “beaded” neurites (bottom) from HC and ADAD 3D-CNs, respectively. Scale bar: 800 nm. FIG. 3E shows representative live-cell FRET images for detecting seed-competent tau from HC and ADAD 3D-CNs at PID26. For each group (HC vs ADAD), the representative images were taken from same field with different channels. n=3 ADAD patients and 3 HC individuals. Scale bar: 25 μm. Data are shown by Mean±SEM. FIG. 3F are immunofluorescence images of p-tau (PHF1) in methanol-fixed 2D cultured cells revealing insoluble tau. Arrow points to the dystrophic tau bead. Scale bar: 25 μm. For quantification in n=4 independent ADAD patients and 4 HC individuals. FIG. 3G shows immunofluorescence of p-tau (AT8) (top) and p-tau+ (PHF1) and K63-ubiquitin+dystrophic neurites (bottom) in ADAD 3D-CNs treated with β-secretase inhibitor IV or DAPT. Arrows depict swelled tau blebs in 3D-CNs. Scale bar: 25 μm. n=3 ADAD patients and 3 HC individuals. * p<0.05 and ** p<0.01 were calculated by unpaired t-test. Adjusted p-values were calculated by one-way ANOVA with Dunnett's multiple comparisons test in G. * adjusted p<0.05 and ** adjusted p<0.01.

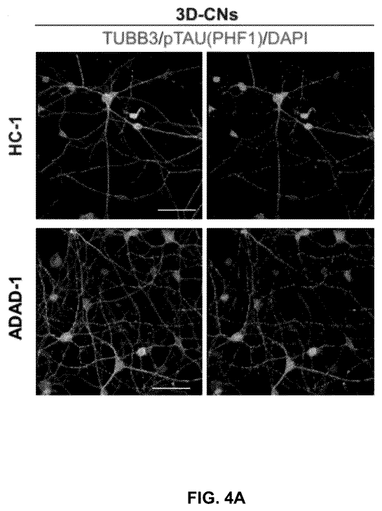

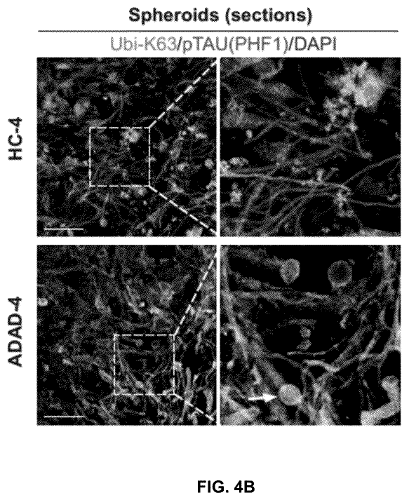

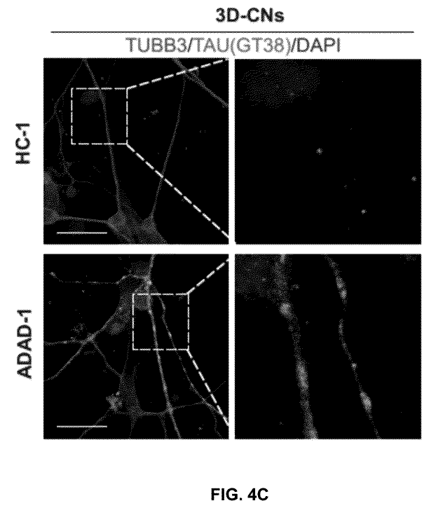

FIGS. 4A-4C provide data from experiments conducted to identify tauopathy in ADAD 3D-CNs and spheroid. FIG. 4A provides representative immunostaining images of p-tau expression in thin gel using PHF1 p-tau antibody. Scale bar: 50 μm. FIG. 4B shows representative images of p-tau (PHF1) and K63-specific ubiquitin immunostaining in the sections of ADAD and HC spheroid. Scale bar: 50 μm. Arrow points to the enlarged tau blebs that are colocalized with K63-linked ubiquitin. FIG. 4C shows immunofluorescence staining images of AD-conformation-specific tau in ADAD and HC neurons using GT-38 tau antibody. Scale bar: 25 μm.

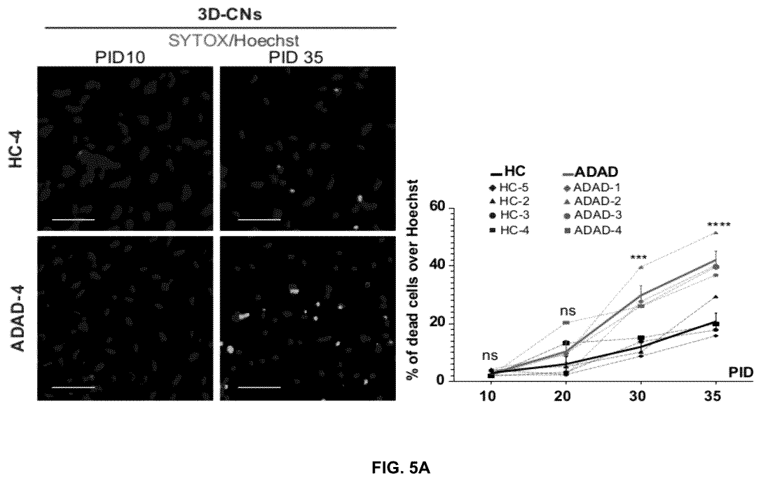

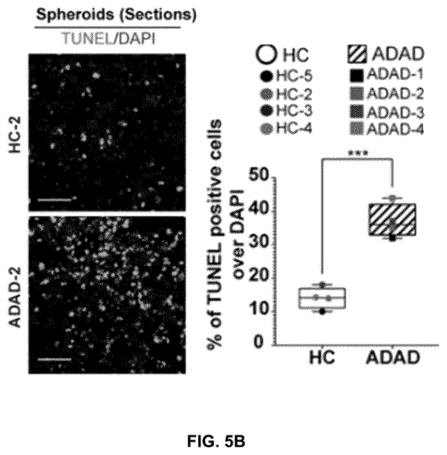

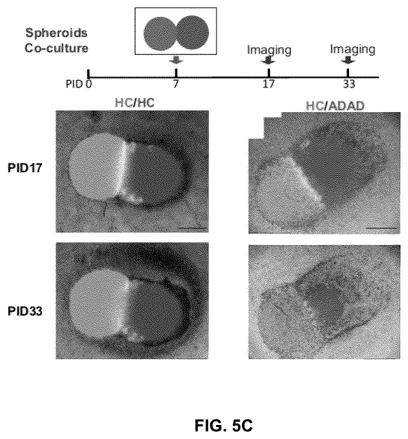

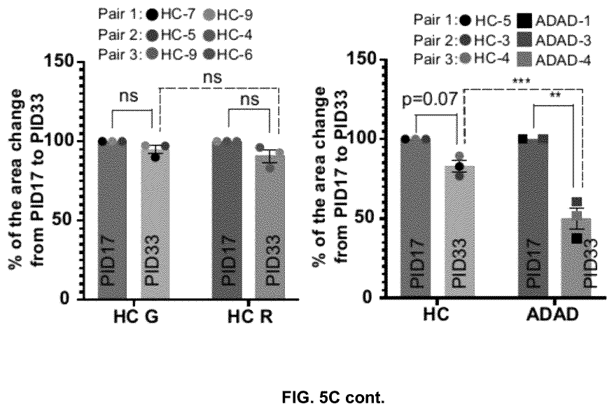

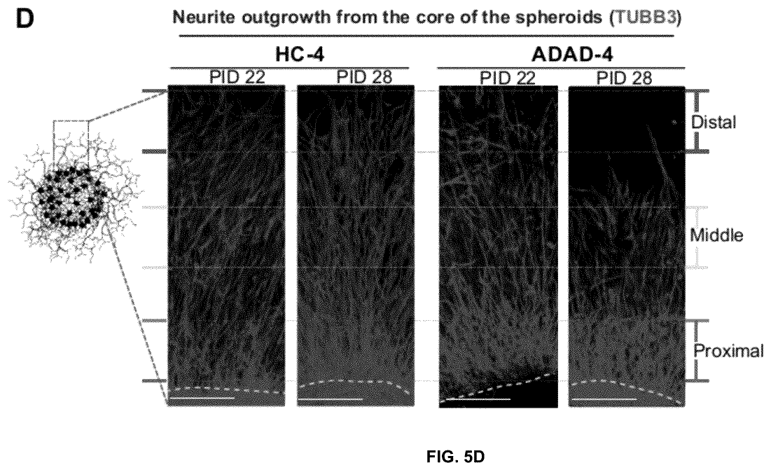

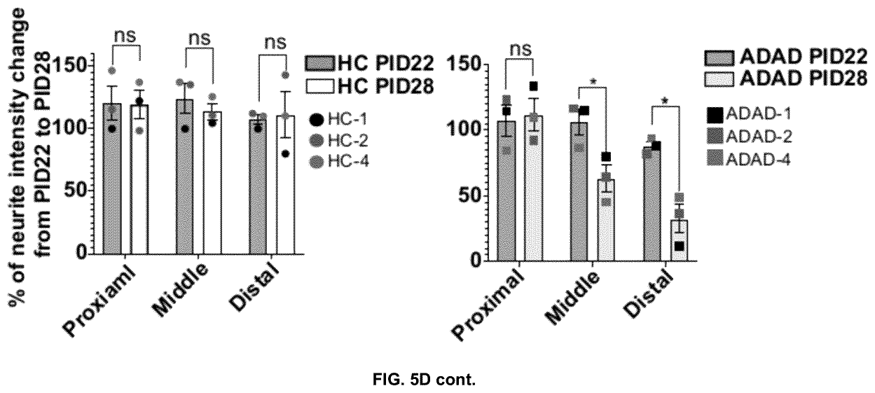

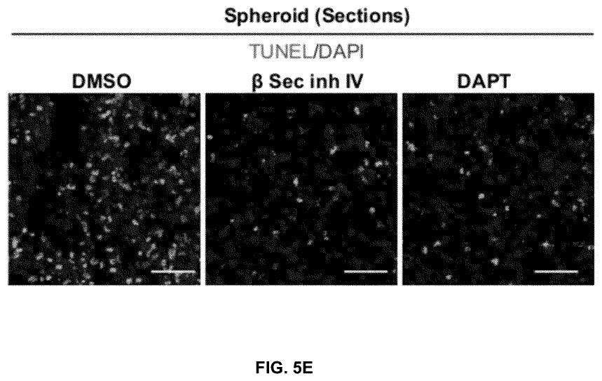

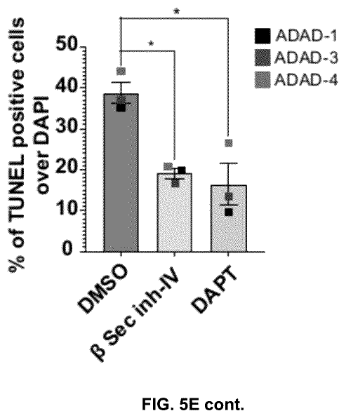

FIGS. 5A-5E show severe neurodegeneration in ADAD 3D-CNs and spheroid. FIG. 5A shows representative immunofluorescence images of Sytox-positive cells in ADAD and HC 3D-CNs at different PIDs. Quantification shows the percentage of Sytox-positive cells over the number of Hoechst-positive cells. n=4 ADAD patients and 4 HC individuals. Solid lines show the average of each group while dotted lines represent each individual. Mean±SEM. p-values were calculated by two-way ANOVA with Šídák's multiple comparisons test. ns=adjusted p>0.05, *** adjusted p<0.001 and **** adjusted p<0.0001. Scale bar: 50 μm. FIG. 5B provides representative images of TUNEL staining in ADAD or HC spheroids sections. Quantifications show the percentage of TUNEL positive cells over DAPI. n=4 ADAD patients and 4 HC individuals. *** p<0.001 by unpaired t-test. Scale bar: 50 μm. FIG. 5C shows stitched live images to display whole spheroids. Two spheroids were labeled by RFP or GFP and co-cultured starting at PID7. At PID17 and PID33, multiple live images were taken and stitched to display the whole spheroid. Left: Two spheroids derived from two different HC individuals are labeled by RFP or GFP. Right: an ADAD spheroids was labeled by RFP and an HC spheroids was labeled with GFP. The areas (μm²) of RFP-labeled and GFP-labeled spheroids at PID17 and PID33 were measured by Imaris. Left (HC/HC co-culture): n=3 pairs (total 5 HC lines). Right (HC/ADAD co-culture): n=3 pairs (3 ADAD patients and 3 HC individuals). Mean±SEM. Adjusted-p values were calculated by two-way ANOVA with Šídák's multiple comparisons test. ns=adjusted p>0.05; ** adjusted p<0.01, and *** adjusted p<0.001. HC G: HC spheroids with GFP; HC R: HC spheroids with RFP. Scale bar: 1 mm. FIG. 5D Left: a cartoon illustration of the typical spheroids with neurite outgrowth from the core. Top: Representative TUBB3 immunostaining images show neurite outgrowth from HC and ADAD spheroids at PID22 and PID28. Yellow dashed lines indicate the border between the core and the neurites. Scale bar: 250 μm. Bottom: Quantifications show the percentage of intensity in proximal, middle, and distal regions of HC and ADAD neurites normalized by the intensity in the same region of PID22 HC-1 neurites (set as 100%). n=3 ADAD patients and 3 HC individuals. Mean±SEM. * p<0.05 was calculated by multiple paired t-tests.

FIG. 5E provides data to show that cell death in ADAD spheroids (PID28) was reduced by treating spheroids with β-secretase inhibitor IV or DAPT starting at PID 16. n=3 ADAD patients. Mean ±SEM. * Adjusted p<0.05 was calculated by one-way ANOVA with Dunnett's multiple comparisons test. Scale bar: 50 μm.

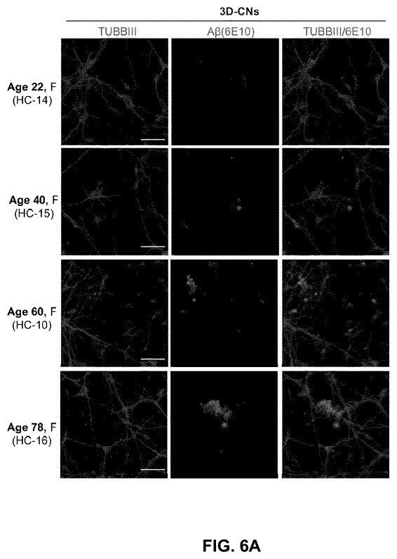

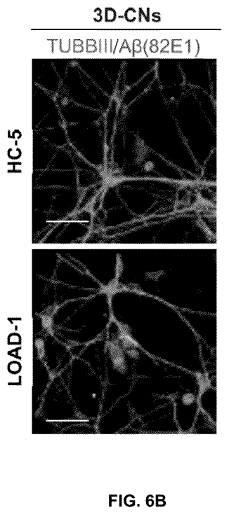

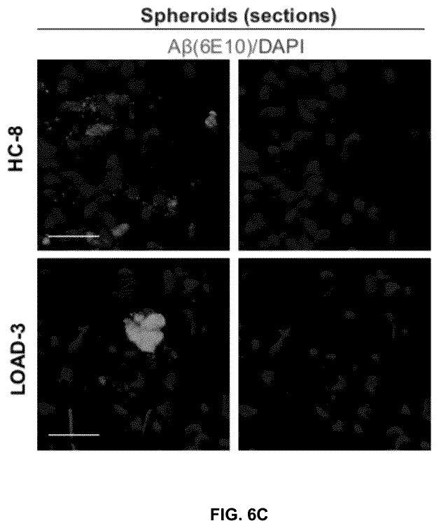

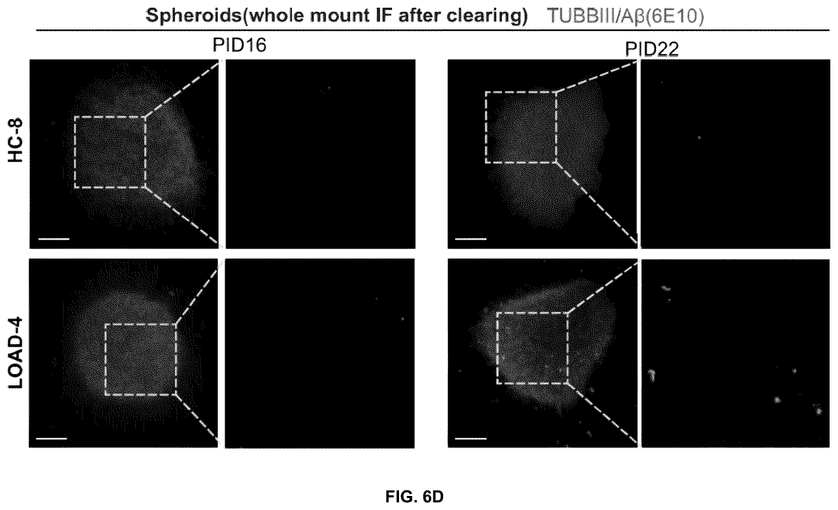

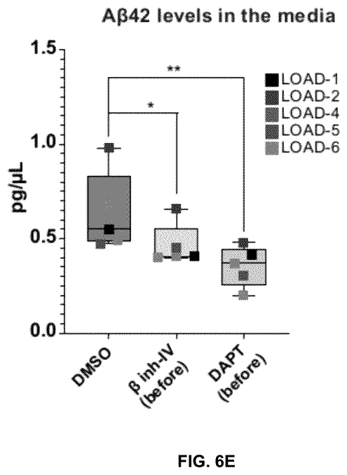

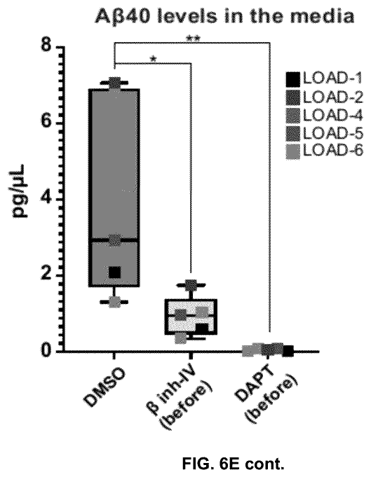

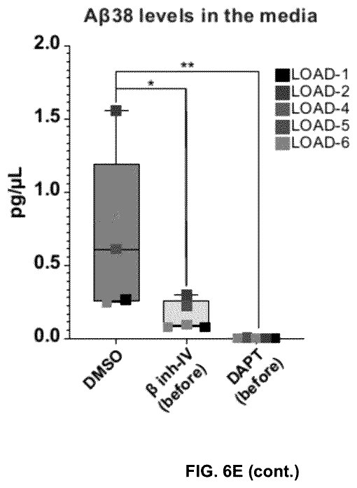

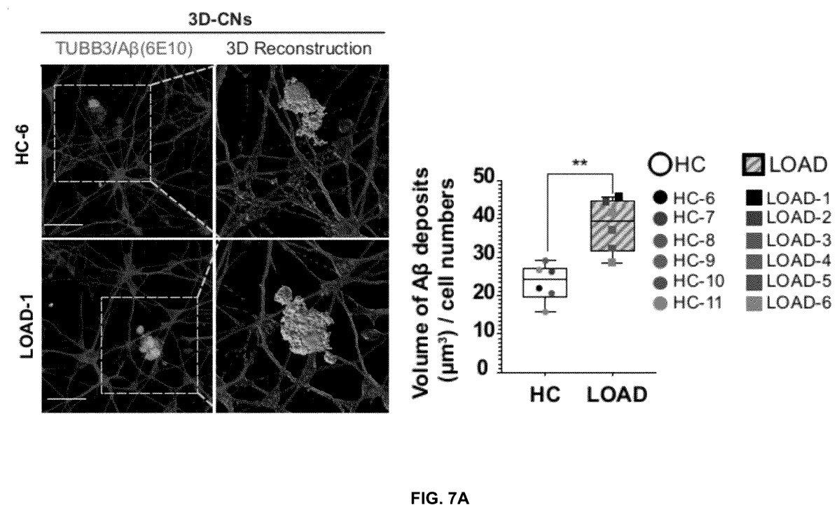

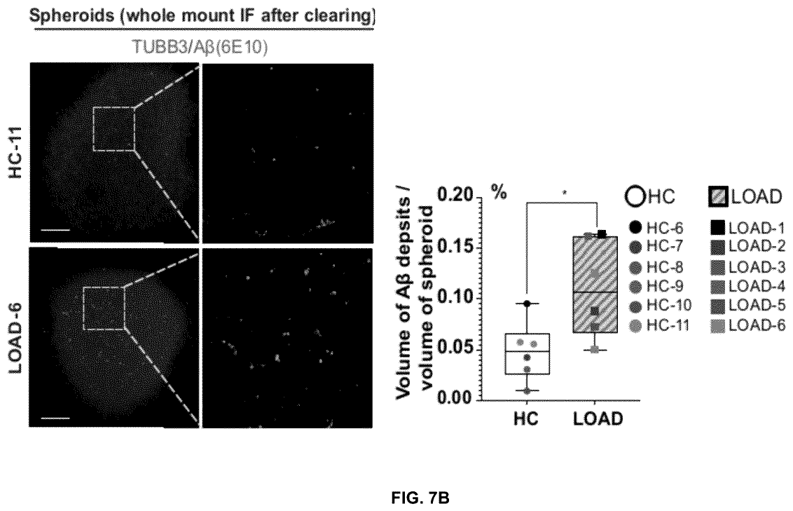

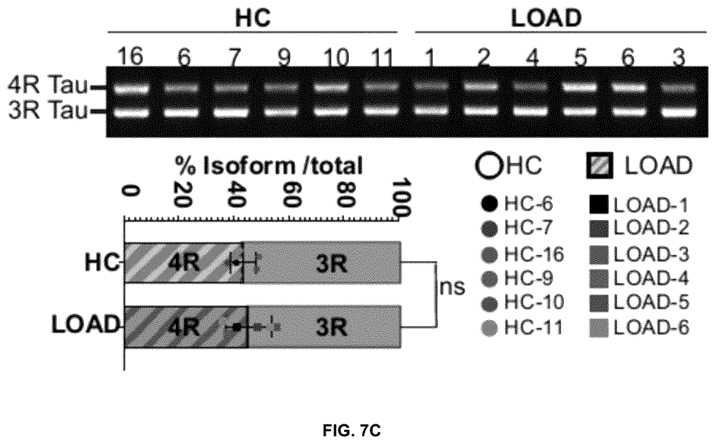

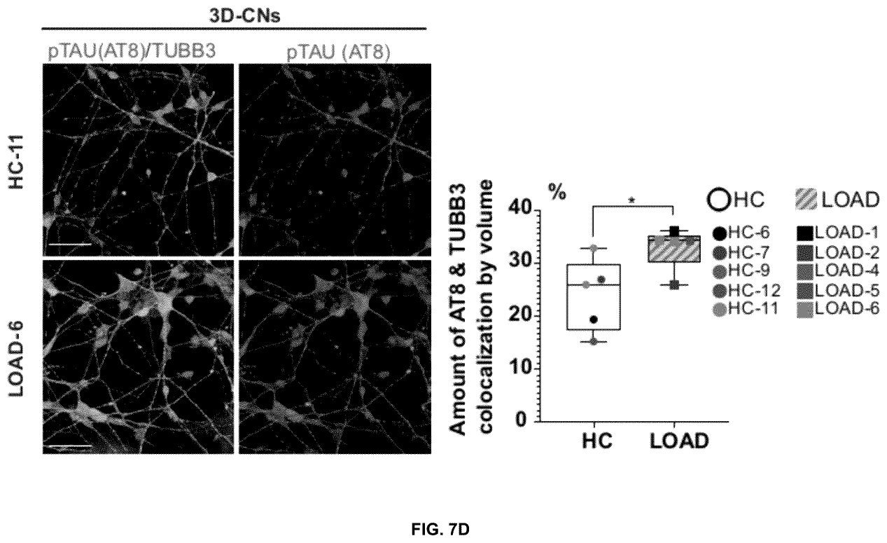

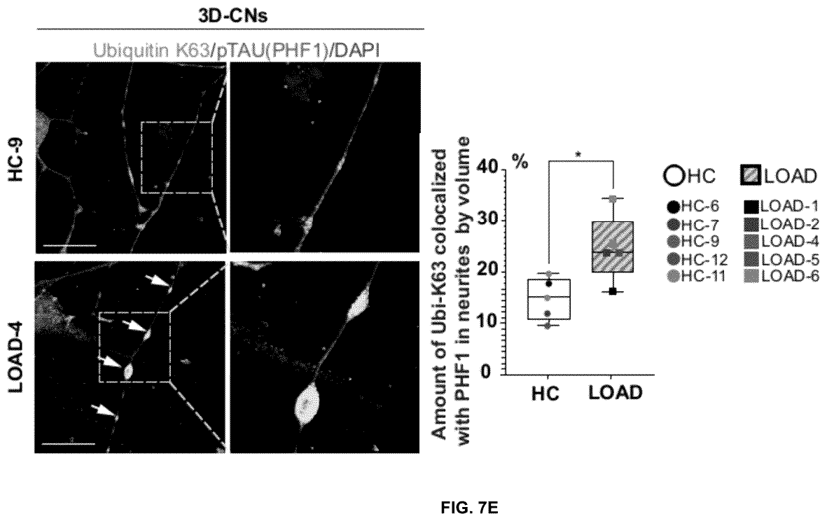

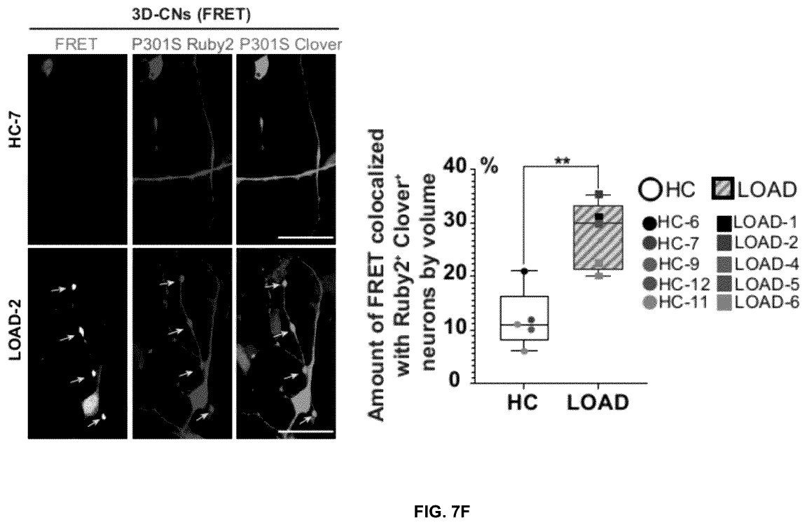

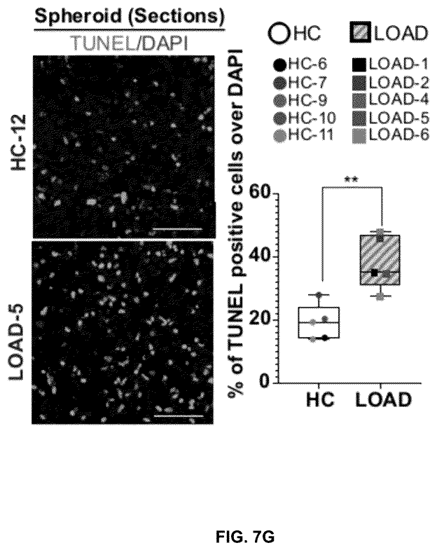

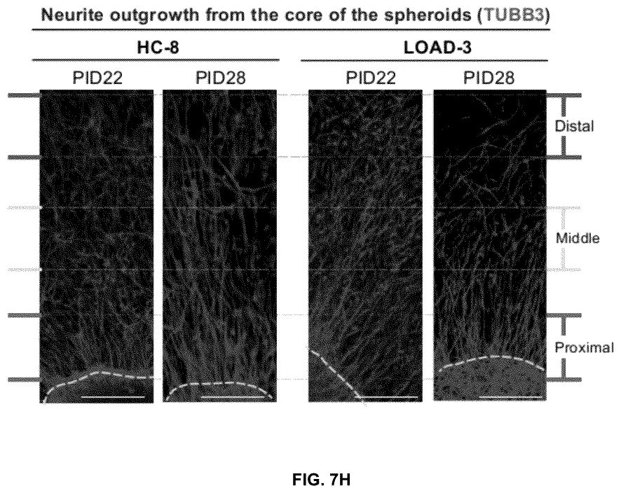

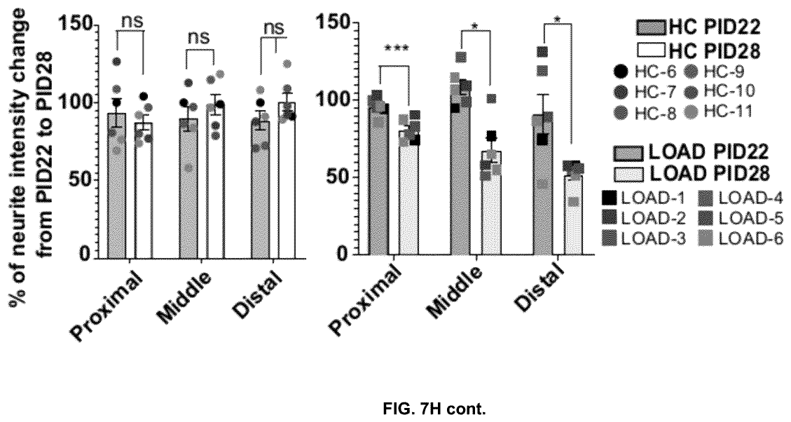

FIGS. 6A-6E show Aβ deposition in aging and LOAD 3D-CNs and spheroids (related with FIG. 7). FIG. 6A provides representative immunostaining images of Aβ deposition (6E10 antibody, green) in 3D-CNs derived from healthy individuals of different ages. F: female. Scale bar: 50 μm. FIG. 6B shows an example of extracellular Aβ deposits labeled by 82E1 Aβ antibody in the thin gel culture of LOAD and HC at PID30. Scale bar: 50 μm. FIG. 6C is representative immunostaining images of Aβ deposits on the sections of PID28 LOAD and HC spheroids using 6E10 Aβ antibody. Scale bar: 25 μm. FIG. 6D provides images with whole mount staining in PID16 and PID22 LOAD and HC spheroids using 6E10 Aβ antibody revealed negligible amount of Aβ deposition in both LOAD and HC group at PID16, but the amount became robust at PID22. Scale bar: 500 μm. FIG. 6E provides mass spectrometry quantification of Aβ42, Aβ40, and Aβ38 detected in the media of PID28 LOAD spheroids that were treated with β-secretase inhibitor IV or DAPT from PID16, corresponding to a time point before the onset of Aβ deposition. n=5 independent LOAD patients. Adjusted p-values were calculated by one-way ANOVA with Dunnett's multiple comparisons test. * p<0.05 and ** p<0.01.

FIGS. 7A-7I show elevated Aβ deposition, pathogenic tau, and neurodegeneration in LOAD 3D-CNs and spheroid. FIG. 7A provides representative immunofluorescence images of Aβ deposits in thin gel culture of PID30 LOAD and HC neurons. Boxed regions were highlighted on the right by 3D reconstruction showing extracellular Aβ deposition. Quantification: total volume (μm³) of Aβ deposits normalized to the total number of cells. Scale bar: 50 μm. FIG. 7B shows whole mount immunostaining with Aβ antibody (6E10) in PID28 LOAD and HC spheroids showed enhanced Aβ deposition in LOAD spheroid. Boxed regions were magnified at the right panels to show Aβ deposition. Volumes (μm³) of total Aβ deposits were normalized to the volumes (μm³) of TUBB3+spheroids for quantification. Scale bar: 500 μm. FIG. 7C provides PCR analysis of 3R and 4R tau isoforms in LOAD and HC 3D-CNs at PID25. Data was shown as Mean±SEM. FIG. 7D shows that at PID30, LOAD 3D-CNs contained significantly more AT8+p-tau than HC 3D-CNs. Scale bar: 50 μm. FIG. 7E shows co-staining of K63-specific ubiquitin (green) and p-tau (PHF1) (red) in the neurites (PID30 3D-CNs). Arrows highlight the swelled dystrophic neurite bulges containing K63-ubiquitin and tau signal in LOAD neurons. Scale bar: 25 μm. FIG. 7F provides live-cell imaging of FRET signal from HC and LOAD 3D-CNs containing Ruby2 and Clover reporters at PID28, and quantification of FRET signals. Scale bar: 25 μm. FIG. 7G shows representative images of TUNEL staining on sections of LOAD and HC spheroids (PID28). Scale bar: 50 μm. FIG. 7H shows the neurite outgrowth at proximal, middle, and distal regions in LOAD and HC spheroids examined by immunofluorescence with TUBB3 antibody. TUBB3 signals in each region of the neurites were compared to same region in HC-6 spheroids at PID22 (set as 100%). Mean±SEM. Scale bar: 250 μm. FIG. 7I shows LOAD spheroids (labeled with RFP) and HC spheroids (labeled with GFP) that were co-cultured at PID7 and fluorescence images were taken at PID17 and PID33. Spheroids size (area, μm²) at PID33 was normalized to its size at PID17. Scale bar: 1 mm. Mean±SEM. FIG. 7I (cont.) shows quantifications: n=6 LOAD patients and 6 HC individuals in A, B, C, H and I, n=5 LOAD patients and 5 HC individuals in D, E, F and G. Unpaired t-tests were used for calculating p-values in A, B, C, D, E, F and G (*p<0.05, ** p<0.01 and ns p>0.05). Multiple paired t-test was calculated for H (p<0.05, *** p<0.001 and ns p>0.05). Two-way ANOVA with Šídák's multiple comparisons test was used for I (*adjusted p<0.05 and **** adjusted p<0.0001).

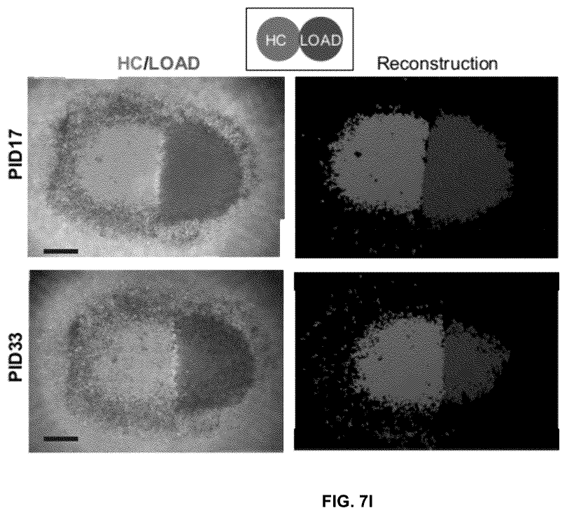

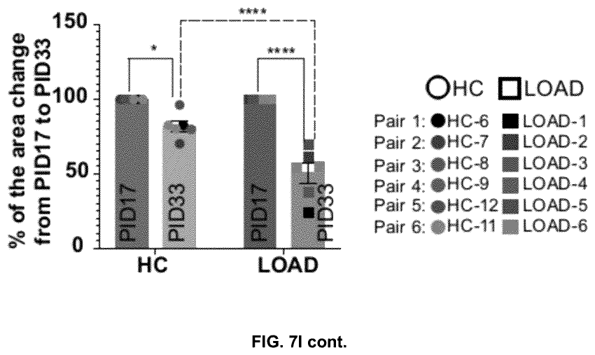

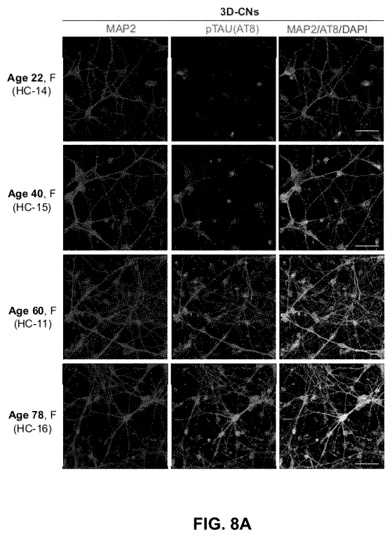

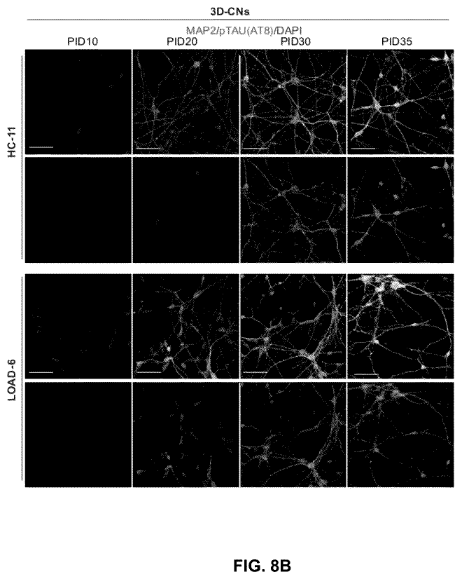

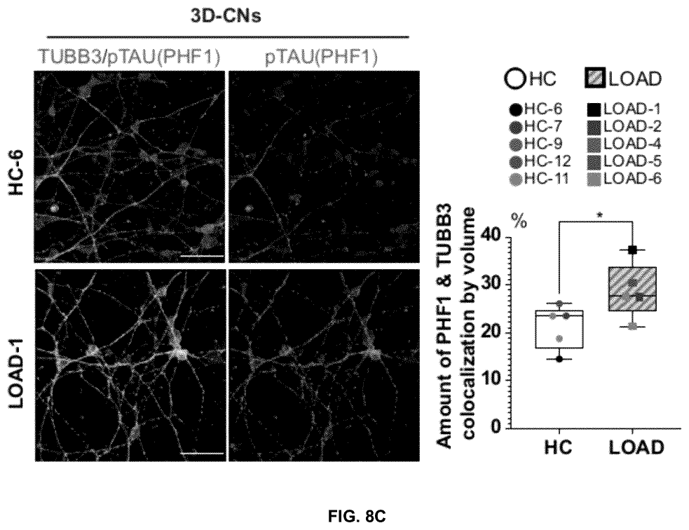

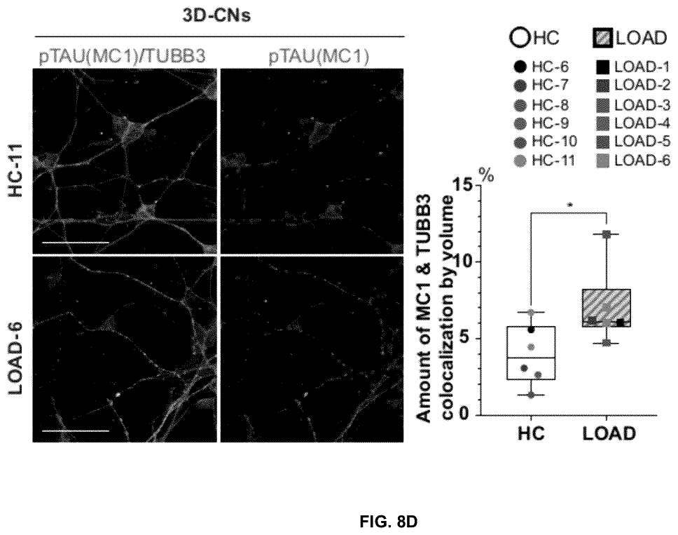

FIGS. 8A-8D show Tau signals in aging and LOAD 3D-CNs (related with FIG. 7). FIG. 8A shows images with immunostaining of p-tau (AT8 antibody, green) in 3D-CNs reprogrammed from healthy individuals of advancing ages. F: female. Scale bar: 50 μm. FIG. 8B shows images of p-Tau immunostaining using AT8 antibody at different PIDs of reprogramming showing p-tau signal at PID30 and PID35 in HC and LOAD 3D-CNs. Scale bar: 50 μm. FIG. 8C provides representative images of p-tau staining using PHF1 antibody in the PID30 LOAD and HC 3D-CNs. n=5 independent LOAD patients and 5 HC individuals. * p<0.05 by unpaired t-test. Scale bar: 50 μm. FIG. 8D Misfolded tau was examined using MC1 tau antibody in both LOAD and HC 3D-CNs at PID30. n=6 independent LOAD patients and 6 HC individuals. * p<0.05 by unpaired t-test. Scale bar: 50 μm.

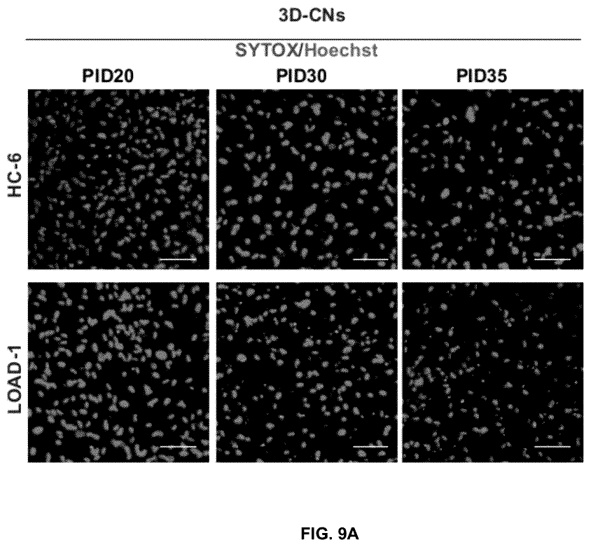

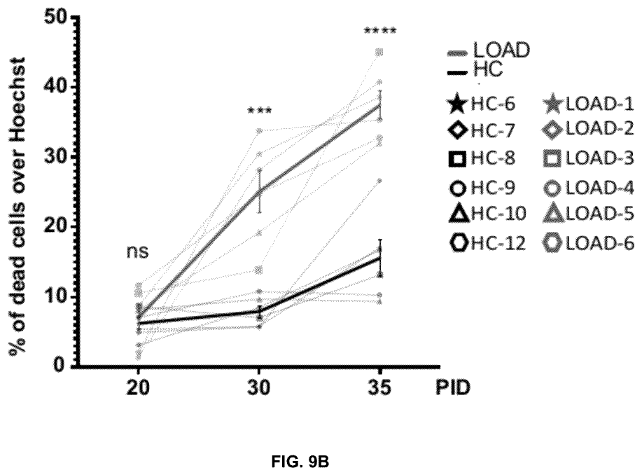

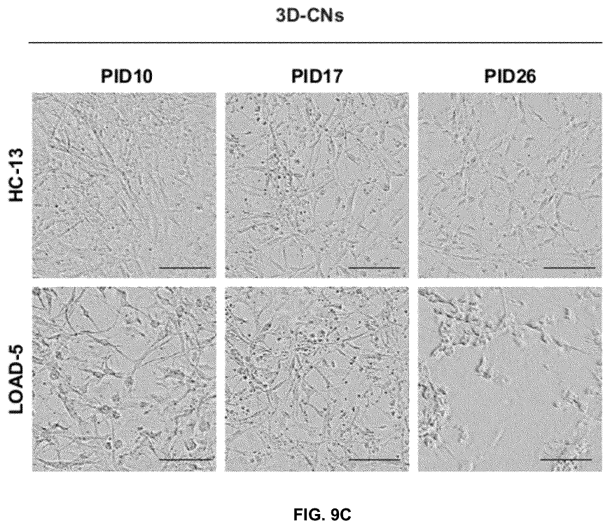

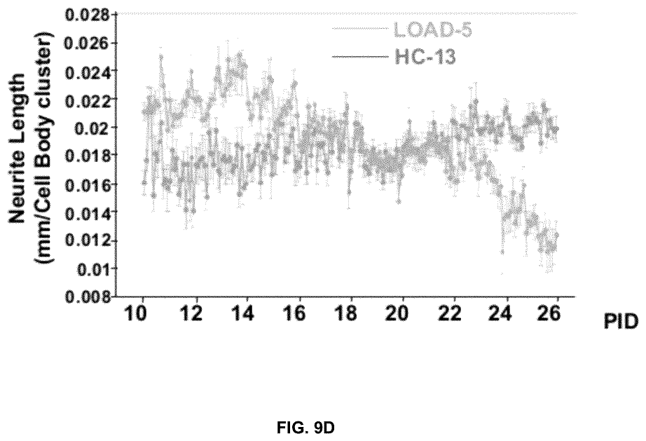

FIGS. 9A-9D provide data showing spontaneous neuronal death in LOAD 3D-CNs (related with FIG. 7). FIG. 9A provides representative images of Sytox staining in LOAD and HC neurons reprogrammed in thin gel at different PIDs. Dead cells were labeled by Sytox-Green and nuclei were stained by Hoechst (blue). FIG. 9B provides statistics showing that the percentage of dead cells was significantly greater in LOAD 3D-CNs at PID30 and PID35 compared to the HC 3D-CNs at same PIDs. n=6 independent LOAD patients and 6 HC individuals. Solid lines show the mean of each group while dotted lines represent each individual. Mean±SEM. p-values were calculated by two-way ANOVA with Šídák's multiple comparisons. ns: adjusted p>0.05, *** adjusted p<0.001, and **** adjusted p<0.0001. Scale bar: 50 μm. FIG. 9C shows live cell imaging of LOAD and HC neurons at different PIDs during neuronal reprogramming. FIG. 9D provides quantification showing the decrease of neurite length in LOAD neurons starting from PID22. Normalized neurite extension was measured by the Neurotracker module of Incucyte Live-Cell analysis. Scale bar: 100 μm.

FIGS. 10A-10C show the effects of inhibiting APP processing in LOAD 3D-CNs and spheroids on tauopathy and neurodegeneration. FIG. 10A. LOAD spheroids were treated with B-secretase inhibitor IV or DAPT starting at PID16 (before the observed onset of Aβ deposition) or PID22 (after the onset of Aβ deposition) and Aβ deposition was examined at PID28 by whole mount immunostaining using Aβ antibody (6E10). Scale bar: 500 μm. FIG. 10B shows images with tau phosphorylation (AT8, top panel) and K63-ubiquitin/p-tau (PHF1). Colocalization (bottom panel) were examined in PID28 LOAD 3D-CNs treated with β-secretase inhibitor IV or DAPT starting at PID16 or PID22. Arrows highlight swelled tau blebs in the bottom panel. Scale bar=25 μm. FIG. 10C shows TUNEL staining of the sections of PID28 LOAD spheroids that were treated with β-secretase inhibitor IV or DAPT starting at PID16 or PID22. Scale bar: 50 μm. For quantifications in A, B, and C, n=5 independent LOAD patients. Imaris was used to calculate the volumes (μm³) or numbers of objects marked by different antibodies. Adjusted p-values were calculated by two-way ANOVA with Šídák's multiple comparisons test. * adjusted p<0.05; ** adjusted p<0.01; **** adjusted p<0.0001.

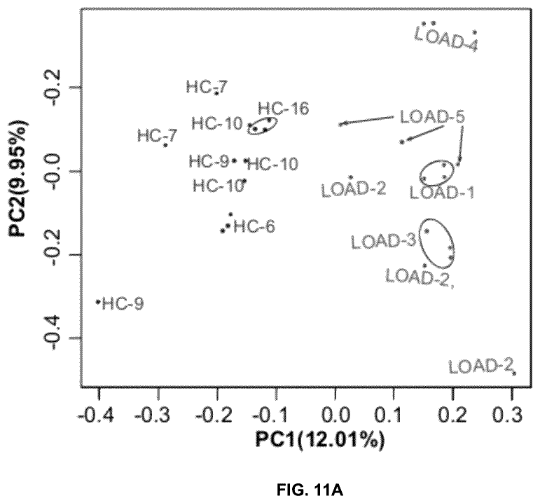

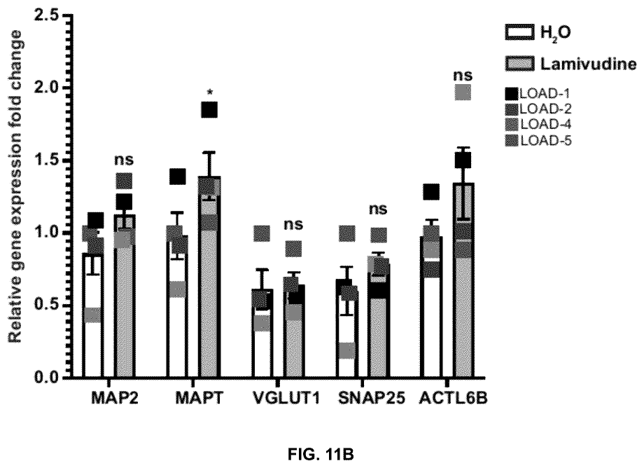

FIGS. 11A-11B show that lamivudine treatment does not dampen the neuronal identity in LOAD spheroids (related with FIG. 6). FIG. 11A provides principal component analysis of gene expression data of LOAD and HC spheroid. A total of 28 samples from 10 independent individuals (5 for LOAD and 5 for HC) were used for analysis. FIG. 11B provides qPCR analysis of the mRNA expression of different neuronal markers in lamivudine treated spheroids compared to control spheroids that were treated with H2O. n=4 LOAD patients. Mean±SEM. ns (no significant difference) and *p<0.05 by multiple unpaired t-tests.

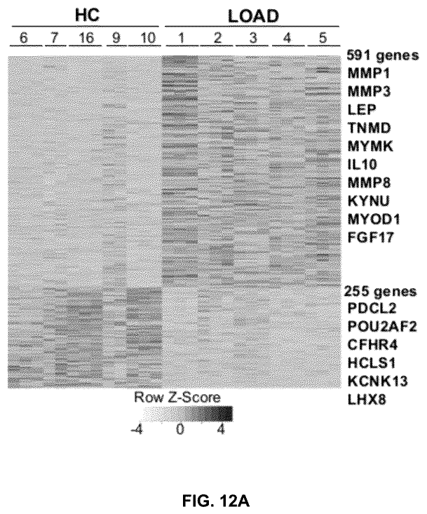

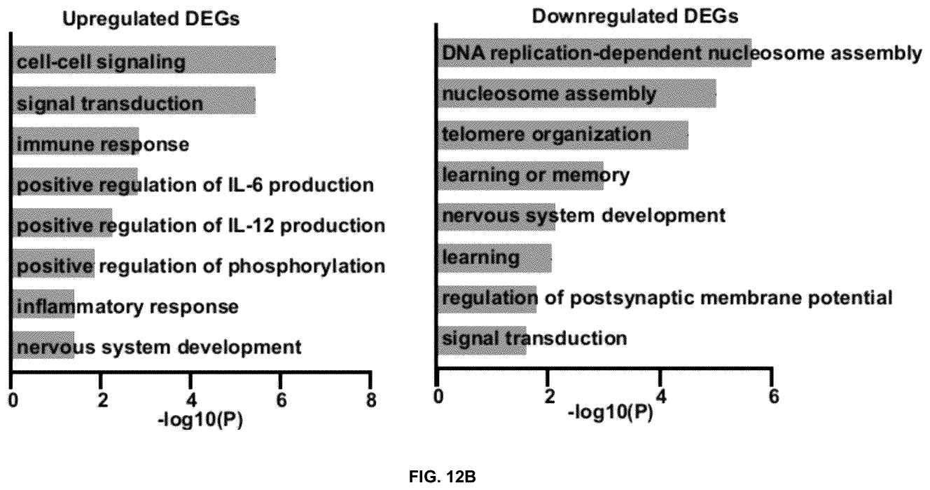

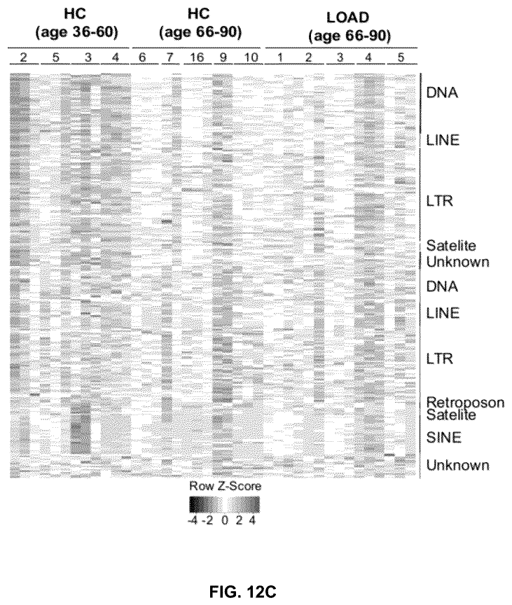

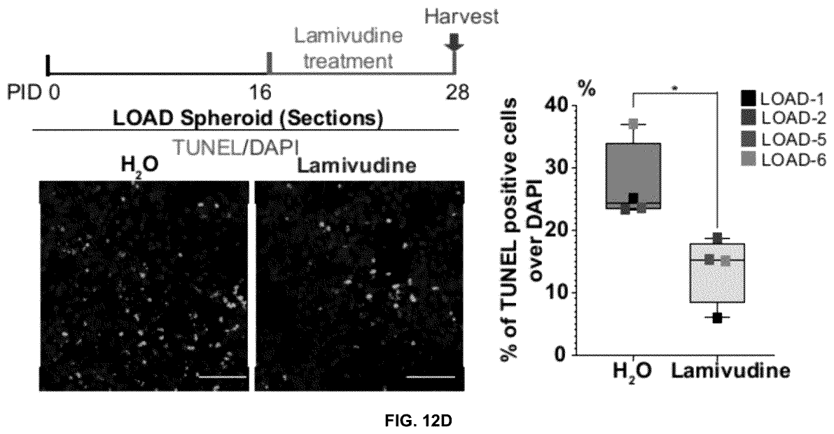

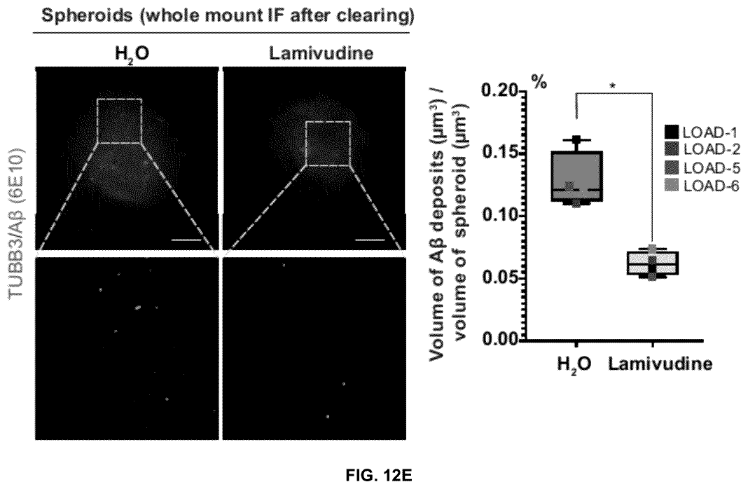

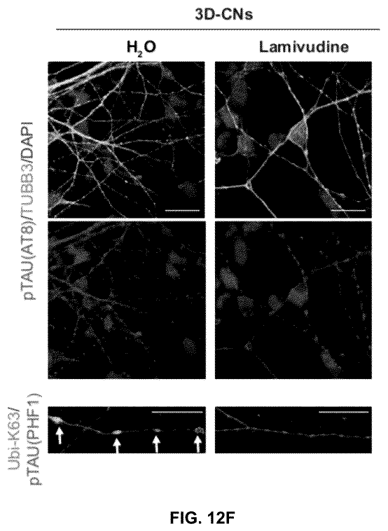

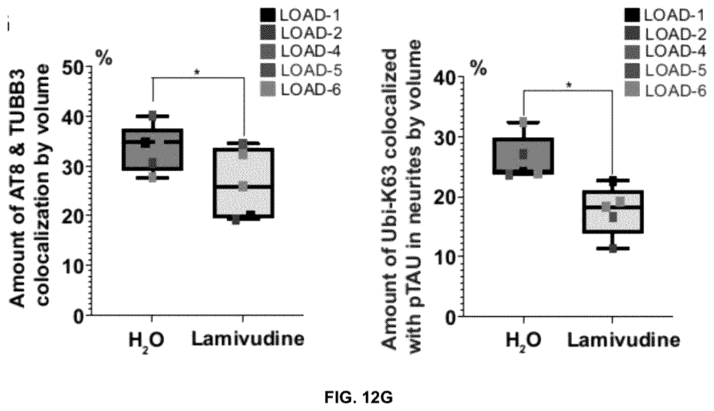

FIGS. 12A-12G show differential gene expression in LOAD spheroids and Lamivudine, a reverse transcriptase inhibitor, ameliorates LOAD neuropathologies. FIG. 12A is a heatmap of gene expression levels of significant (p<0.05, |log 2FC|>0.58) differentially expressed genes (DEGs) in PID25 LOAD and HC spheroids by RNA-seq. A total of 28 samples from 10 independent individuals (5 for LOAD and 5 for HC) were used for analysis. FIG. 12B provides Top Gene Ontology (GO) terms associated with upregulated (left) and downregulated (right) DEGs analyzed by DAVID analysis. FIG. 12C is a heatmap of significant (p<0.05, |log 2FC|>0.58) differentially expressed transposable elements (DETEs) in PID25 LOAD and HC spheroids analyzed by RNA-seq. FIG. 12D. Top: Schematic of lamivudine treatment for inhibiting RTE formation in LOAD spheroid. Bottom images: TUNEL staining of the dead cells (green color) in lamivudine- or H2O-(control) treated spheroids (PID28). Scale bar: 50 μm. FIG. 12E is representative whole mount immunostaining images of Aβ deposition in lamivudine and H2O-treated spheroids at PID28. Scale bar: 500 μm. FIG. 12F p-Tau labeled by AT8 antibody (top panel) and co-localization of p-tau (PHF1) and K63-ubiquitin (bottom panel) in lamivudine or H2O-treated LOAD 3D-CNs at PID28. Arrows highlight the dystrophic swelled tau blebs. Scale bar=25 μm. FIG. 12G provides quantification of FIG. 12F. Percentages of p-tau (AT-8) colocalized with TUBB3 (left) and K63-linked ubiquitin colocalized with p-tau (PHF1) in neurites (right) of LOAD 3D-CNs treated with lamivudine or H2O. For quantifications: n=4-5 LOAD patient lines in D, E, and G. * p<0.05 was calculated by paired t-test.

### DETAILED DESCRIPTION

The present disclosure is based in part on the surprising discovery that Alzheimer's disease (AD) patient-derived fibroblasts can be effectively reprogrammed into cortical neurons and self-assembled neuronal spheroids in three-dimensional (3D)-cultures using microRNA-mediated direct neuronal reprogramming of fibroblasts. Interestingly, these reprogrammed neuronal cells exhibit most of the age characteristics of the fibroblasts, while also recapitulating the neuropathological characteristics of AD neurons.

Advances in stem cell and reprogramming technologies have enabled the generation of various human cell types, including neurons differentiated from patient-specific induced pluripotent stem cells (iPSCs). Although these iPSC-derived neurons hold great promise for modelling and studying neurological development and diseases, induction of pluripotency in somatic cells erases the age signatures of the donor cells, resulting in iPSC-derived neurons reflecting embryonic identity. This imposes challenges for modelling age-associated diseases such as LOAD, as it does not account for human age as the major contributing risk factor. For example, healthy adult human neurons express 4-repeat (4R) and 3-repeat (3R) tau isoforms at approximately 1:1 ratio. However, iPSC-derived neurons only express marginal levels of 4R tau isoforms, similar to fetal neurons. iPSC-derived neurons from AD patients exhibit increased levels of Aβ42, active GSK-3B, phosphorylated tau, endosomal abnormalities, and oxidative stress, but no late-stage neuropathological features, such as neurofibrillary tau tangles and neurodegeneration. Therefore, establishing a patient-derived neuronal system that retains the age signature from the donor is critical to recapitulate the key neuropathologic events in AD.

Direct cell lineage reprogramming converts cells of interest from one lineage to another through forced expression of genetic factors. By bypassing the pluripotent induction and stem cell stages, direct reprogramming offers experimental advantages to aging-related disorders, since age signatures such as the epigenetic clock, telomere length, and transcriptomic changes are propagated from the starting cells to the reprogrammed cells. Brain-enriched microRNAs, can bring about neurogenic reprogramming effectors that directly convert human fibroblasts into neurons. These miRNAs can bring about chromatin reconfiguration, allowing for sequential steps of fibroblast identity erasure, followed by neuronal program activation. The miRNAs-induced neuronal fate can synergize with additional transcription factors (TFs) to guide the conversion to disease-relevant neuronal subtypes with high efficiency, and may be suitable for studying neuron-intrinsic properties of aging or age-dependent pathology.

As disclosed herein, the feasibility of modeling the neuropathology of AD by generating reprogrammed cortical neurons, while addressing the question of whether the age-maintained, patient-derived neurons would be sufficient to manifest key neuronal pathologies of AD was tested. Leveraging the high efficiency miRNAs-mediated chromatin remodeling and reprogramming, a 3D MATRIGEL™-based cell culture system for generating aged cortical neurons and self- organized neuronal spheroids to capture secreted Aβ and facilitate plaque formation was developed. 3D-cultured neurons and spheroids reprogrammed from both ADAD and LOAD patients endogenously exhibited extracellular Aβ deposits, tau dysregulation, and neurodegeneration.

Surprisingly, inhibiting Aβ formation in LOAD neurons and spheroids reprogrammed from fibroblasts reduced Aβ deposition, tauopathy and neuronal cell death, demonstrating that the toxic effect of Aβ accumulation can be captured by these induced neurons and spheroids. By RNA-sequencing (RNA-seq), neuroinflammation was identified as a top enriched pathway in LOAD spheroids. Lastly, inhibition of transposable element synthesis alleviated AD pathologies in patient-derived neurons and spheroid. These results demonstrate the robustness of the disclosed 3D culture-based direct neuronal reprogramming of patient fibroblasts to capture key neuropathological features of AD.

The disclosed neurons and spheroids reprogrammed from both familial AD (FAD) and LOAD patients exhibited AD-like neuronal phenotypes, including extracellular Aβ deposition, dystrophic neurites with hyperphosphorylated, K63-ubiquitin-positive, seeding-competent tau, and spontaneous neuronal death in culture. Moreover, treatment with β- or γ-secretase inhibitors in LOAD patient-derived neurons and spheroids before Aβ deposit formation significantly lowered Aβ formation, as well as tauopathy and neurodegeneration. However, the same treatment after the cells already formed Aβ deposits only exhibited a mild effect. Additionally, inhibiting the synthesis of age-associated retrotransposable elements (RTEs) by treating LOAD neurons and spheroids with the reverse transcriptase inhibitor, lamivudine, alleviated AD neuropathology. Thus, the present disclosure demonstrates that direct neuronal reprogramming of AD patient fibroblasts in a 3D environment can capture age-related neuropathology and reflect the interplay between Aβ accumulation, tau dysregulation, and neuronal death. Moreover, miRNA-based 3D neuronal conversion may be used to identify compounds that can potentially ameliorate AD-associated neurodegeneration.

The compositions and method disclosed herein demonstrate the effectiveness and sufficiency of 3D-cultured, patient-derived cortical neurons through miRNAs-mediated direct reprograming for modeling critical neuropathological hallmarks of AD. This approach presents opportunities to investigate molecular events intrinsic to neurons that drive AD-associate neurodegeneration and may serve as a patient-specific neuron platform for testing various compounds or target genes for personalized therapeutic interventions. The technology described herein enables the generation of cortical neurons directly from patients, enabling disease modeling, and drug screening while simultaneously providing a source of patient specific cells for regenerative medicine. The present disclosure contributes to the fields of developmental biology, regenerative medicine, direct conversion, neuroscience, genetics, chromatin biology, and microRNA biology.

### I. Reprogrammed Cortical Neurons and Neuronal Spheroid

Disclosed herein is a reprogrammed cortical neuron or neuronal spheroid, generated from a patient-derived somatic cell by expressing at least one miRNA, or at least one exogenous transcription factor, or any combination thereof in the patient-derived somatic cell; and culturing the patient-derived somatic cell in a 3D-culture, thereby reprogramming the somatic cell to a neuronal cell. Also provided herein are methods of generating the reprogrammed cortical neuron or neuronal spheroids.

The disclosed somatic cell may be any somatic cell suitable for 3D-culture into neuronal cells. The somatic cell may be a keratinocyte, a fibroblast cell, a peripheral blood cell, a peripheral mononuclear blood cell, a urinary cell, or a bone marrow cell. The somatic cell may be a derived from the same AD patient for whom a treatment is sought, referred herein as autologous. The somatic cell may be derived from a patient, different from the AD patient for whom treatment is sought. The somatic cell may be derived from an age matched AD patient. The somatic cell may be derived from a patient at least 50, at least 55, at least 60, at least 65, at least 70, at least 75, at least 80, at least 85, at least 90, at least 95, at least 100 or more years in age. The somatic cell may be a fibroblast. The fibroblast may be derived from any suitable tissue from a subject, for example, a dermal fibroblast, a cardiac fibroblast, a pulmonary fibroblast, an intestinal fibroblast and synovial fibroblast. The fibroblast may be a dermal fibroblast. The fibroblast may be derived from an AD fibroblast cell line. Non-limiting examples of AD fibroblast cell lines include AG06840, UCL 33, UCL 53, UCL 803, FA15-552, FA18-634, FA12-453, AG05810, FA17-623, FA12-463, FA18-633, AG06869. The fibroblast may be a derived from a patient who has or is suspected of having Alzheimer's disease. The fibroblast may be a derived from the same AD patient for whom a treatment is sought, referred herein as autologous. The fibroblast may be derived from a patient, different from the AD patient for whom treatment is sought. The fibroblast may be derived from an age matched AD patient. The fibroblast may be derived from a patient at least 50, at least 55, at least 60, at least 65, at least 70, at least 75, at least 80, at least 85, at least 90, at least 95, at least 100 or more years in age.

The somatic cell may comprise at least one ectopic or exogenously expressed micro RNA (miRNA) that is involved in re-programming an adult somatic cell (e.g. a fibroblast) to a neuronal fate. The miRNAs capable of reprogramming somatic cells into neurons may coordinate epigenetic and transcriptional changes resulting in neuronal cell fate conversion; induce a generic neuronal state characterized by the loss of fibroblast identity, the presence of a pan-neuronal gene expression program, and absence of subtype specificity; initiate subunit switching within BAF chromatin remodeling complexes while separately repressing the neuronal cell-fate inhibitors REST, Co-REST, and SCP1; or alter the expression of genes involved in DNA methylation, histone modifications, chromatin remodeling, and chromatin compaction.

The at least one miRNA as described herein can concertedly and separately target components of genetic pathways that antagonize neurogenesis and promote neuronal differentiation during neural development; open the neurogenic potential of adult human fibroblasts and thus provide a platform for subtype-specific neuronal conversion of human cells; orchestrate widespread neuronal chromatin reconfiguration; or promote the opening of neuronal subtype-specific loci.

The microRNA (miRNA) may regulate genetic pathways by binding to their target transcripts and repressing their expression. Target specificity can be governed largely through short sequence complementarity within the 5′ end of a miRNA, enabling a single miRNA to target hundreds of mRNA transcripts. Moreover, a single mRNA can be targeted by multiple miRNAs, markedly enlarging the effect on single gene repression. These attributes position miRNAs to affect broad changes in gene expression and genetic programs despite their limited size. Thus, the somatic cell may comprise a nucleic acid sequence encoding at least one or more of the miRNAs. The somatic cell may express at least one, or at least two, or at least three, or at least four, or at least five, at least six, or at least seven, or at least eight, or at least nine, or at least ten or more different exogenous miRNA involved in re-programming a somatic cell to a neuronal fate.

The convergence of genetic controls by miRNAs towards a specific biological process is exemplified by miR-9/9 *- and miRNA-124 miRNAs activated at the onset of neurogenesis. For most microRNAs, upon association with the RISC complex, only one of the two arms, either the 5′ or 3′, is preferentially selected to generate the processed miRNA (sometimes called guide strand), while the other tends to be used more infrequently (passenger strand or star strand). In the case of miR-9 genes, the guide strand can be generated either from the 5′ (miR-9-5p, heretofore referred to as miR-9) or the 3′ arm (miR-9-3p, heretofore referred to as miR-9*) depending on the gene considered. Together, miR-9 and miR-9* are referred to as miR9/9*. Similarly, for miR-124, the same polynucleotide sequence can encode 2 different miRNAs (miR-124-5p and miR-124-3p). As used herein, miR-124 may include individual or both mature products of miR-124, i.e. miR-124-5p and miR-124-3p. The miR-9/9* and miR-124 can synergistically act as a molecular switch to initiate subunit switching within BAF chromatin remodeling complexes while separately repressing the neuronal cell-fate inhibitors REST, Co-REST, and SCP1. miR-9/9* and miR-124 target components of genetic pathways that antagonize neurogenesis to promote a neuronal identity during development. Together, miR-9/9* and miR-124 are referred herein as miR-9/9 *-124.

Thus, the miRNA supporting reprogramming of the disclosed somatic cells to a neuronal lineage may comprise miR-9, miR-9*, or miR-124 or any combination thereof. The miRNA supporting reprogramming of the disclosed somatic cells to a neuronal lineage may comprise miR-9, miR-9*, and miR-124 (i.e., miR-9/9 *-124). The miRNA may be encoded by a polynucleotide sequence comprising the nucleic acid sequence set forth in SEQ ID NO: 1, and SEQ ID NO: 4, or a complementary sequence thereof, or a sequence at least about 80% identical thereto. In an aspect, the miRNA may comprise a nucleic acid sequence set forth in any one of SEQ ID NO: 2, SEQ ID NO: 3, SEQ ID NO: 5 or SEQ ID No: 6, or a sequence at least about 90% identical thereto.

TABLE A
miRNA encoding sequences and
the encoded miRNAs.

| SEQ | | |
|---|---|---|
| ID | | |
| NO: | NAME | SEQUENCE |
| 1 | DNA | CGGGGTTGGTTGTTATCTTTGGTTATCTAGCTGT |
| | sequence | ATGAGTGGTGTGGAGTCTTCATAAAGCTAGATAA |
| | encoding | CCGAAAGTAAAAATAACCCCA |
| | miRNA-9 | |
| 2 | mi-RNA9 | UCUUUGGUUAUCUAGCUGUAUGA |
| 3 | mi-RNA 9* | AUAAAGCUAGAUAACCGAAAGU |
| 4 | DNA | AGGCCTCTCTCTCCGTGTTCACAGCGGACCTTGA |
| | sequence | TTTAAATGTCCATACAATTAAGGCACGCGGTGAA |
| | encoding | TGCCAAGAATGGGGCTG |
| | miRNA-124 | |
| 5 | MIR124-1 | CGUGUUCACAGCGGACCUUGAU |
| 6 | MIR124-1 | UAAGGCACGCGGUGAAUGCC |

In addition to showing the surprising capability of miRNAs, such as miR-9/9 *-124, in coordinately stimulating the reconfiguration of chromatin accessibilities, it is further shown herein that the microRNA-induced neuronal state may be co-induced, supported and/or maintained by exogenous expression of one or more transcription factors. The disclosure reveals a modular synergism between microRNAs and transcription factors that allow lineage-specific neuronal reprogramming, providing a platform for generating cortical neurons. Thus, the current disclosure also encompasses the somatic cell further comprising at least one neurogenic transcription factor The neurogenic transcription factor may be any transcription factor capable of inducing, supporting and/or maintaining the neuronal fate and or neuronal spheroids nature of the cells.

The somatic cell may comprise at least one exogenous neurogenic transcription factor in addition to the disclosed miRNA. The somatic cell may comprise at least one, or at least two, or at least three, or at least four, or at least five, at least six, or at least seven, or at least eight, or at least nine, or at least ten or more different exogenous transcription factor(s) involved in re-programming a somatic cell to a neuronal fate. The transcription factors as described herein can be administered by any method known in the art. For example, transcription factors can be provided exogenously or expressed ectopically. Non-limiting examples of suitable neurogenic transcription factors that may induce, support or help maintain cortical-like neurons include CTIP2, DLX1, DLX2, NEUROD2, ASCL1 and MYT1L. The neurogenic factor may be any one or more of NEUROD2, ASCL1 and MYT1L. Thus, the somatic cell may further comprise a nucleic acid sequence encoding at least one neurogenic transcription factor. The somatic cell may comprise a nucleic acid sequence encoding an amino acid sequence set forth in any one of SEQ ID NO: 7, SEQ ID NO: 8, or SEQ ID NO: 9, or a derivative thereof, or an amino acid sequence at least about 60% identical thereto.

| SEQ | NAME: | |
|---|---|---|
| ID | Uniprot | |
| NO: | ID | SEQUENCE |
| 7 | Human | MLTRLFSEPGLLSDVPKFASWGDGEDDEPRSDKGDAPPPPPPAPGPGAPGPARAAKPVPL |
| | NEUROD2: | RGEEGTEATLAEVKEEGELGGEEEEEEEEEEGLDEAEGERPKKRGPKKRKMTKARLERSK |
| | Q15784 | LRRQKANARERNRMHDLNAALDNLRKVVPCYSKTQKLSKIETLRLAKNYIWALSEILRSG |
| | | KRPDLVSYVQTLCKGLSQPTTNLVAGCLQLNSRNFLTEQGADGAGRFHGSGGPFAMHPYP |
| | | YPCSRLAGAQCQAAGGLGGGAAHALRTHGYCAAYETLYAAAGGGGASPDYNSSEYEGPLS |
| | | PPLCLNGNFSLKODSSPDHEKSYHYSMHYSALPGSRPTGHGLVFGSSAVRGGVHSENLLS |
| | | YDMHLHHDRGPMYEELNAFFHN |
| 8 | Human | MESSAKMESGGAGQQPQPQPQQPFLPPAACFFATAAAAAAAAAAAAAQSAQQQQQQQQQQ |
| | ASCL1: | QQAPQLRPAADGQPSGGGHKSAPKOVKRORSSSPELMRCKRRLNFSGFGYSLPQQQPAAV |
| | P50553 | ARRNERERNRVKLVNLGFATLREHVPNGAANKKMSKVETLRSAVEYIRALQQLLDEHDAV |
| | | SAAFQAGVLSPTISPNYSNDLNSMAGSPVSSYSSDEGSYDPLSPEEQELLDFTNWF |
| 9 | Human | MEVDTEEKRHRTRSKGVRVPVEPAIQELFSCPTPGCDGSGHVSGKYARHRSVYGCPLAKK |
| | MYTIL: | RKTQDKQPQEPAPKRKPFAVKADSSSVDECDDSDGTEDMDEKEEDEGEEYSEDNDEPGDE |
| | Q9UL68 | DEEDEEGDREEEEEIEEEDEDDDEDGEDVEDEEEEEEEEEEEEEEEENEDHQMNCHNTRI |
| | | MQDTEKDDNNNDEYDNYDELVAKSLLNLGKIAEDAAYRARTESEMNSNTSNSLEDDSDKN |
| | | ENLGRKSELSLDLDSDVVRETVDSLKLLAQGHGVVLSENMNDRNYADSMSQQDSRNMNYV |
| | | MLGKPMNNGLMEKMVEESDEEVCLSSLECLRNQCFDLARKLSETNPQERNPQONMNIRQH |
| | | VRPEEDFPGRTPDRNYSDMLNLMRLEEQLSPRSRVFASCAKEDGCHERDDDTTSVNSDRS |
| | | EEVFDMTKGNLTLLEKAIALETERAKAMREKMAMEAGRRDNMRSYEDQSPROLPGEDRKP |
| | | KSSDSHVKKPYYGKDPSRTEKKESKCPTPGCDGTGHVTGLYPHHRSLSGCPHKDRVPPEI |
| | | LAMHESVLKCPTPGCTGRGHVNSNRNSHRSLSGCPIAAAEKLAKAQEKHQSCDVSKSSQA |
| | | SDRVLRPMCFVKOLEIPQYGYRNNVPTTTPRSNLAKELEKYSKTSFEYNSYDNHTYGKRA |
| | | IAPKVOTRDISPKGYDDAKRYCKDPSPSSSSTSSYAPSSSSNLSCGGGSSASSTCSKSSF |
| | | DYTHDMEAAHMAATAILNLSTRCREMPQNLSTKPQDLCATRNPDMEVDENGTLDLSMNKQ |
| | | RPRDSCCPILTPLEPMSPQQQAVMNNRCFOLGEGDCWDLPVDYTKMKPRRIDEDESKDIT |
| | | PEDLDPFQEALEERRYPGEVTIPSPKPKYPQCKESKKDLITLSGCPLADKSIRSMLATSS |
| | | QELKCPTPGCDGSGHITGNYASHRSLSGCPRAKKSGIRIAQSKEDKEDQEPIRCPVPGCD |
| | | GQGHITGKYASHRSASGCPLAAKROKDGYLNGSQFSWKSVKTEGMSCPTPGCDGSGHVSG |
| | | SFLTHRSLSGCPRATSAMKKAKLSGEQMLTIKORASNGIENDEEIKOLDEEIKELNESNS |
| | | QMEADMIKLRTQITTMESNLKTIEEENKVIEQQNESLLHELANLSQSLIHSLANIQLPHM |
| | | DPINEQNFDAYVTTLTEMYTNQDRYQSPENKALLENIKQAVRGIQV |

The nucleic acid sequence encoding the at least one miRNA and/or the at least one neurogenic transcription factors may further comprise one or more regulatory elements. As used herein, “regulatory elements” refers to any sequence element that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary, or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that comprises an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

The present disclosure provides for reprogrammed cortical neurons and neuronal spheroids obtained from reprogramming patients fibroblast cells by ectopic expression of microRNAs, and/or transcription factors, and uses thereof as models of disease progression. The disclosed somatic cell may be re-programmed into a cortical neuron or neuronal spheroid. The reprogrammed cortical neuron may resemble any known cortical neuron known in the art. The cortical neuron may be a pyramidal neuron, an interneuron, a stellate cell, a betz cell or a bipolar cell. The reprogrammed cortical neuron may resemble a hippocampal pyramidal neuron or a neocortical pyramidal neuron. The reprogrammed cortical neuron may comprise at least one of the miRNA disclosed herein, and optionally at least one of the transcription factors disclosed herein.

Neuronal spheroids are three-dimensional (3D) cell cultures composed of neurons and other supporting cells that self-organize into spherical structures resembling certain aspects of the brain's organization. These spheroids are commonly used in neuroscience research to study various aspects of neural development, function, disease modeling, and drug testing. The disclosed neuronal spheroids cells may comprise at least one of the miRNA disclosed herein, and optionally at least one of the transcription factors disclosed herein.

These directly converted neurons or neuronal spheroids may retain age-associated marks of starting adult human somatic cells (e.g. fibroblasts), including the epigenetic age (also known as the epigenetic clock), oxidative stress, DNA damage, miRNAome, telomere lengths and transcriptome. This unique feature offers potential advantages in modeling adult-onset disorders using directly converted neurons.

Thus, the disclosed reprogrammed cortical neuron or neuronal spheroids may comprise one or more age signatures of the patient-derived somatic cell. The age signatures may be epigenetic changes, telomere length, or transcriptomic changes or combinations thereof. Epigenetic changes may include changes in chromatin accessibility, loss of histones and heterochromatin, aberrant histone modifications, and deregulated expression/activity of miRNAs. Transcriptomic changes may include changes in global gene expression, cell type-specific expression, gene co-expression networks, expression quantitative trait loci, and alternative splicing. The induced neuronal cells may exhibit decreased synaptic function and increased susceptibility, similar to similarly aged patient neurons. Additionally, the reprogrammed neurons may exhibit expression changes in various types of noncoding RNAs, including microRNAs (miRNAs), long noncoding RNAs (lncRNAs), and circular RNAs (circRNAs). Methods of determining epigenetic changes, telomere length changes, and transcriptomic changes are well known in the art and may include RNA-seq, mRNA microarrays, in-situ hybridization, 3D-sequencing, PCR, qPCR, quantitative trait locus (QTL) analysis, single cell transcriptomics, protein profiling, western blot analysis, and mass spectrometric studies.

The disclosed reprogrammed cortical neuron or neuronal spheroids may further comprise one or more age-related LOAD neuropathologies of the patient. The one or more age-related LOAD neuropathologies may comprise extracellular Aβ deposition, dystrophic neurites, hyperphosphorylated, K63-ubiquitin-positive tau, seed-competent tau, spontaneous neuronal death, increased levels of Aβ42, active GSK-3B, phosphorylated tau, endosomal abnormalities, oxidative stress, or any combination thereof. Methods of detecting neuropathologies in a neuron are well known in the art. Exemplary methods include immunostaining, immunofluorescence techniques, microscopy, western blotting, mass spectrometry and related techniques.

### II. Methods

Disclosed herein is a method of generating the reprogrammed cortical neuron or neuronal spheroids from the patient-derived somatic cell. The method may comprise providing the patient-derived somatic cell expressing at least one of the miRNA, or at least one of the exogenous transcription factor, or any combination thereof in the patient-derived somatic. The method may further comprise culturing the patient-derived somatic cell in a 3D-culture, thereby reprogramming the somatic cell to a neuronal cell. The neuronal cell may be a cortical neuron. The method may further comprise developing a neuronal spheroids in the 3D-culture.

Direct 3D reprogramming of cells involves the conversion of one type of differentiated cell directly into another specific cell type within a three-dimensional (3D) culture environment, bypassing the intermediate step of inducing pluripotency. This approach aims to directly convert one cell type into another without reverting to a pluripotent state, thereby potentially avoiding limitations associated with induced pluripotent stem cell (iPSC) reprogramming. The current disclosure comprises culturing the somatic cell in a 3D-culture to induce direct 3D reprogramming to neuronal cells.

The disclosed method may comprise initially growing the somatic cells (for example a patient-derived fibroblast or a fibroblast cell line) in a liquid culture. The somatic cell may be a derived from a patient who has or is suspected of having Alzheimer's disease. The somatic cell may be a derived from an autologous AD patient. The somatic cell may be derived from a patient, different from the AD patient for whom treatment is sought. The somatic cell may be derived from an age matched AD patient. The somatic cell may be derived from a patient at least 50, at least 55, at least 60, at least 65, at least 70, at least 75, at least 80, at least 85, at least 90, at least 95, at least 100 or more years in age.

Protocols for isolating and culturing fibroblasts are well known in the art and any suitable protocol may be used with the current method. For example, the fibroblasts may be seeded in a fibroblast medium (FM) comprising 15% fetal bovine serum (FBS): Dulbecco's Modified Eagle Medium (DMEM), with high glucose containing 15% FBS, 0.1% 55 mM beta-mercaptoethanol, 1% IM HEPES buffer, 1% nonessential amino acids, 1% sodium pyruvate, 1% GlutaMAX and 1 penicillin/streptomycin solution. Any one or more of these components may be replaced with a suitable equivalent and the concentrations of the components can be adjusted as necessary.

The somatic cell may be transduced with a vector comprising the nucleic acid sequence encoding the at least one miRNA and optionally, the at least one neuronal transcription factor as disclosed herein. The vector may comprise an isolated nucleic acid, a plasmid vector, a transposon, or a viral vector. The vector may be a viral vector. Examples of suitable a viral vector include an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. The disclosed viral vector may be a lentiviral vector. The vector may comprise the nucleic acid sequence as set forth in any one of SEQ ID NO: 1 and SEQ ID NO: 4. The vector may comprise the nucleic acid sequence encoding at least one miRNA corresponding to any one or more of SEQ ID NO: 2, SEQ ID NO: 3, SEQ ID NO: 5, and SEQ ID NO: 6. The vector may comprise a nucleic acid sequence encoding at least one neurogenic transcription factor comprising the amino acid sequence set forth in any one of SEQ ID NOs: 7-9. The vector may further comprise at least one or more regulatory elements disclosed herein. Methods of administering a vector into a cell are well known in the art and may include viral transduction methods, non-viral transduction methods including lipofection, calcium phosphate transfection, electroporation, particle bombardment, microinjection or sonoporation. Alternatively, the miRNA and/or the neurogenic transcription factor may be delivered to the somatic cells using nanoparticles or liposomes.

The disclosed somatic cell can then be grown in a 3D culture. Use of a three-dimensional matrix, preferably a gel, results in reprogramming and expansion of the reprogrammed neuronal cells and development of neuronal spheroid. Cells thrive in natural 3D biopolymer networks like collagen and MATRIGEL™ (trade name for a gelatinous protein mixture secreted by Engelbreth-Holm-Swarm mouse sarcoma cells), as well as in engineered materials with motifs for adhesion and enzymatic degradation that facilitate cell growth and migration. Structured and reinforced hydrogels have also been developed to improve perfusion characteristics. Hydrogels with embedded carbon foam have been used to improve mechanical stability. A suitable three-dimensional matrix may comprise collagen I, hyaluronic acid, alginate, fibrin, gelatin, PEG based hydrogels. The three-dimensional matrix may comprise an extracellular matrix from the Engelbreth-Holm-Swarm tumor or any component thereof such as laminin, collagen, preferably type 4 collagen, entactin, and optionally further heparan-sulfated proteoglycan or any combination thereof. One example of such a matrix is a matrix comprising MATRIGEL™ The matrix may comprise by weight about 60-85% laminin, 5-30% collagen IV, optionally 1-10% nidogen, optionally 1-10% heparan sulfate proteoglycan and 1-10% entactin. MATRIGEL™ solid components usually comprise approximately 60% laminin, 30% collagen IV, and 8% entactin. Entactin is a bridging molecule that interacts with laminin and collagen. The three-dimensional matrix may further comprise growth factors, such as any one of EGF (epidermal growth factor); FGF (fibroblast growth factor); NGF; PDGF; IGF (insulin-like growth factor), which may be IGF-1; TGF-β; and tissue plasminogen activator. The three-dimensional matrix may also be free of any of these growth factors. The matrix may comprise a concentration of about 1 mg/ml to about 10 mg/ml of the matrix solid substance (e.g. MATRIGEL™) in a suitable medium. The matrix may be mixed with the somatic cells and medium to achieve a final concentration typically ranging from 1-5 mg/ml. The optimal matrix concentration may vary depending on the cell type and experimental goals. The volume of matrix solution used per well or culture dish may depend on the desired thickness of the gel layer and the number of cells seeded. Exemplary method of developing cortical neurons and spheroids are also provided herein in the examples.

The reprogrammed neurons or spheroids may be used for research and/or therapeutic purposes. For example, the disclosed method may be adapted for screening for a drug for treatment of Alzheimer's disease. The method of screening may be used for personalized medicine or for generic screening for drugs for Alzheimer's disease. The screening method may comprise providing a patient-derived somatic cell; expressing at least one miRNA, or at least one exogenous transcription factor, or any combination thereof in the somatic cell; and culturing the somatic cell in a 3D-culture as disclosed herein to generate a reprogrammed cortical neuron cell or neuronal spheroid. The reprogrammed cortical neuron cell or neuronal spheroids may be contacted with the drug to obtain a treated reprogrammed cortical neuron cell or neuronal spheroid, which can be analyzed for the level of at least one AD-related neuropathology. The levels of the at least one AD-related pathology can then be compared between treated and control samples; wherein reduction in the levels of at least one AD-related neuropathology in the treated reprogrammed cortical neuron cell or neuronal spheroids in comparison to a control level is indicative of the effectiveness of the drug for treating Alzheimer's disease. The control level can be obtained by analyzing the level of the AD neuropathology in untreated reprogrammed cortical neuron cell or neuronal spheroids from the same patient, or a different patient, or a patient population. The at least one AD related neuropathology may comprise extracellular Aβ deposition, dystrophic neurites, hyperphosphorylated, K63-ubiquitin-positive tau, seed-competent tau, spontaneous neuronal death, increased levels of Aβ42, active GSK-3B, phosphorylated tau, endosomal abnormalities, oxidative stress, or any combination thereof. Suitable methods for analyzing cellular neuropathology are known in the art and are disclosed herein. The method can be adapted for testing a new therapeutic agent, a known therapeutic agent, determining efficacy levels, determining dosage of therapeutics to balance cellular toxicity with efficacy, and for determining suitable personalized treatment options for a subject in need thereof. The screening method may be used for screening a single drug or in a high-throughput screen.

A “therapeutic agent” can be any agent that effects a desired clinical outcome in a subject having AD or LOAD, suspected of having AD or LOAD, and/or likely to develop or acquire AD or LOAD. In an aspect, a disclosed therapeutic agent can be an oligonucleotide therapeutic agent. A disclosed oligonucleotide therapeutic agent can comprise a single-stranded or double-stranded DNA, gRNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

A therapeutic agent can be a “drug” and means a molecule, group of molecules, complex or substance administered to an organism for diagnostic, therapeutic, preventative medical, or veterinary purposes. The disclosed method can be used to screen the effectiveness of known drugs for treatment of AD. Non-limiting examples of known drugs for the treatment of Alzheimer's disease include brexpiprazole, donepezil, rivastigmine, galantamine, tacrine, memantine or lamivudine. The screen may also be used to determine the feasibility of repurposing a known drug for use in the treatment of AD.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The following definitions and methods are provided to better define the present invention and to guide those of ordinary skill in the art in the practice of the present invention. Unless otherwise noted, terms are to be understood according to conventional usage by those of ordinary skill in the relevant art.

As used in the specification and the appended claims, the singular forms “a,” “an,” and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including,” “containing,” or “characterized by,” and is inclusive or open-ended. “Comprising” does not exclude additional, unrecited components or steps.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

The terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the” and “said” are intended to mean that there are one or more of the elements. The terms “comprising,” “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology,” “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order: aligned, Trec rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and Val to Ile or Leu.

As used herein, “operably linked” means that expression of a gene is under the control of a regulatory element with which it is spatially connected. In some instance the regulatory element is a promoter and when used in this context, the expression of a gene is under control of the promoter with which it is spatially connected. A regulatory element (e.g., a promoter) can be positioned 5′ (upstream) or 3′ (downstream) of a gene under its control. The distance between the regulatory element (e.g., promoter) and a gene can be approximately the same as the distance between that regulatory element (promoter) and the gene it controls in the gene from which the regulatory element (e.g., promoter) is derived. As is known in the art, variation in this distance can be accommodated without loss of function (i.e., without loss of promoter function).

As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary, or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that comprises an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

The term “transduction”, as used herein, is a process by which foreign DNA is introduced into a cell (e.g., by a virus, viral vector, bacteriophage, naked DNA). Transduction methods are well known; see e.g., Transduction, Genetic at the US National Library of Medicine Medical Subject Headings (MeSH). Except as otherwise noted herein, therefore, the process of the present disclosure can be carried out in accordance with such processes. For example, as described herein, miRNAs and transcription factors can be cloned into a viral vector (e.g., a lentivirus plasmid, Sendai virus). For example, after cloning into the viral vector, a virus (e.g., lentivirus) is produced, and the fibroblasts are infected. The virus then integrates its genome (containing the miRNAs and TFs) into the fibroblast genome. As such, these ectopic genes are stably expressed by the transduced cells. A viral vector can be any viral vector known in the art. For example, the viral vector can be a retrovirus, a lentivirus, an adenovirus, or an adeno-associated virus.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “patient” can refer to a subject that has been diagnosed with or is suspected of having an APOE associated disease or condition. In an aspect, a patient can refer to a subject that has been diagnosed with or is suspected of having Alzheimer's disease (AD) or late-onset Alzheimer's disease (LOAD). In an aspect, a patient can refer to a subject that has been diagnosed with or is suspected of having AD such as for example, LOAD, and is seeking treatment or receiving treatment for AD or LOAD.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Development of 3D Direct Neuronal Reprogramming of AD Fibroblasts into Cortical Neurons (CNs) and Spheroid

Fibroblast samples from 10 AD patients (4 ADAD and 6 LOAD) and 16 age- and sex-matched healthy control (HC) donors (Table 1) were reprogrammed with neurogenic effectors miR-9/9 *-124, and TFs NEUROD2 and MYT1L to drive the conversion towards cortical neurons.

TABLE 1
Subject profile

| Sample | Fibroblast | | Age at | Age at | | |
|---|---|---|---|---|---|---|
| ID | ID | Sex | biopsy | onset | Genotype | Origin |
| ADAD-1 | AG06840 | male | 56 | | ADAD, PSEN1 A246E | Coriell |
| HC-1 | AG04148 | male | 56 | | Healthy Control | Coriell |
| ADAD-2 | UCL 33 | male | 58 | 49 | ADAD, APP V717I | UCL |
| Group | AG04453 | male | 52 | | Healthy Control, | Coriell BLSA |
| HC-2 | | | | | | |
| ADAD-3 | UCL 53 | male | 38 | | ADAD, PSEN1 M146I | UCL |
| Group | AG04060 | male | 44 | | Healthy Control | Coriell |
| HC-3 | | | | | | |
| ADAD-4 | UCL 803 | female | 42 | 38 | ADAD, APP V717F | UCL |
| HC-4 | AG05838 | female | 36 | | Healthy Control | Coriell |
| Group | AG08260 | male | 61 | | Healthy Control | Coriell |
| HC-5 | | | | | | |
| LOAD-1 | FA15-552 | male | 89 | | Biomarker Positive, | Knight ADRC |
| | | | | | LOAD, APOE3/4 | |
| HC-6 | FA18-634 | male | 89 | | Biomarker Negative, | Knight ADRC |
| | | | | | Healthy, APOE3/3 | |
| LOAD-2 | FA12-453 | female | 72 | | Biomarker Positive, | Knight ADRC |
| | | | | | LOAD, APOE4/4 | |
| HC-7 | AG13246 | female | 66 | | Healthy Control | Coriell |
| LOAD-3 | AG05810 | female | 79 | | LOAD, APOE3/4 | Coriell |
| HC-8 | AG11020 | female | 79 | | Healthy Control | Coriell |
| LOAD-4 | FA17-623 | female | 87 | | Biomarker Positive, | Knight ADRC |
| | | | | | LOAD, APOE3/3 | |
| HC-9 | FA12-463 | female | 83 | | Biomarker Negative, | Knight ADRC |
| | | | | | Healthy, APOE3/3 | |
| LOAD-5 | FA18-633 | male | 70 | | Biomarker Positive, LOAD | Knight ADRC |
| HC-10 | AG14251 | male | 68 | | Healthy Control | Coriell |
| LOAD-6 | AG06869 | female | 60 | | LOAD | Coriell |
| HC-11 | AG08379 | female | 60 | | Healthy Control | Coriell |
| HC-12 | AG13369 | male | 68 | | Healthy Control | Coriell |
| HC-13 | AG04356 | male | 74 | | Healthy Control | Coriell |
| HC-14 | GM02171 | female | 22 | | Healthy Control | Coriell |
| HC-15 | AG07307 | female | 40 | | Healthy Control | Coriell |
| HC-16 | AG11246 | female | 78 | | Healthy Control | Coriell |

The reprogrammed cells were electrically active in 2D culture, showing robust inward and outward currents and multiple action potentials, as measured by whole-cell recording (FIG. 1L). To limit the diffusion of secreted Aβ in conventional 2D culture and maximize Aβ deposition a MATRIGEL™-based 3D-culture system was developed consisting of a thin gel to allow for high resolution imaging of reprogrammed neurons and self-assembled neuronal spheroid. For thin gel culture, cells were dissociated at post-induction day (PID) 7 and resuspended in 15% MATRIGEL™, followed by 2-3 weeks of reprogramming to generate 3D-cultured cortical neurons (3D-CNs) (FIG. 1A). During reprogramming, 3D-CNs underwent morphological changes from fibroblasts to neurons (FIG. 1B and FIG. 1M) and showed increased expression of long genes, a transcriptomic feature unique to neurons (FIG. 1N). 3D-CNs also showed decreased expression of fibroblast genes and upregulation of neuronal genes during reprogramming (FIG. 10). Immunostaining revealed that 3D-CNs derived from both HCs and AD patients expressed neuronal markers including NCAM1, MAP2, and SLC17A7 (also known as VGLUT1) (FIG. 1C and FIG. 1P).

For generating neuronal spheroid, cells were dissociated at PID7, pelleted, and transferred into a transwell insert to form one neuronal spheroid, followed by 2-3 weeks of culture in neuronal media (FIG. 1D). Reprogrammed spheroids exhibited extensive neurite outgrowth from the core of the spheroids (FIG. 1E). By 3-4 weeks, neuronal spheroids derived from HCs and AD patients displayed reduced expression of fibroblast marker genes and increased expression of long genes and neuronal markers compared to their starting fibroblasts, as assessed by RNA-seq (FIGS. 1Q and 1R) and confirmed by qPCR of neuronal markers such as MAP2, MAPT (also known as TAU), SNAP25, ACTL6B (also known as BAF53B, a marker of mature neurons), SLC17A6 (also known as VGLUT1, a glutamatergic neurons marker), TBR1 (a cortical marker), and POU3F2 (also known as BRN2, a cortical marker) (FIG. 1S). Immunofluorescence staining of PID28 spheroids sections revealed robust expression of NCAM1, MAP2 and NEUN in HC and AD spheroids (FIG. 1F). Collectively, these results confirm the neuronal identity of reprogrammed cells in 3D-direct reprogramming conditions.

### Example 2. Aβ Deposition in FAD 3D-CNs and Spheroid

To assess AD-like neuronal phenotypes, immunostaining analyses of 3D-CNs derived from ADAD patient and age- and sex-matched HC individual using N-terminus specific Aβ antibody 82E1 was performed. Extracellular Aβ deposits with diameters of 10-50 μm were detected by 82E1 Aβ antibody in ADAD 3D-CNs at PID30 (FIG. 1G and FIG. 2A). Aβ deposition in the extracellular space of 3D-CNs from multiple ADAD patients with mutations in PSEN1 or APP (Table 1) and age- and sex-matched HC fibroblast lines using another anti-Aβ antibody 6E10 was examined. Similar to the result of using 82E1 antibody, the extracellular deposition of Aβ in ADAD 3D-CNs was also detected by 6E10 Aβ antibody, with a threefold increase of extracellular Aβ deposit volume compared to HC 3D-CNs (FIG. 1H). To examine the Aβ deposition in spheroid, the Aβ42 level through electrochemiluminescence assay in spheroids after 4 weeks of reprogramming was first measured. In contrast to low Aβ42 levels detected in HC spheroid, ADAD spheroids showed significantly higher levels of Aβ42 (FIG. 1I), consistent with the impact of ADAD mutations on APP processing. Next, whole mount immunostaining was performed using Aβ antibody (6E10) on PID22 and PID28 spheroids followed by clearing of the spheroids to allow 3D fluorescent imaging. At both PID22 and PID28, ADAD spheroids exhibited a significantly higher amount of Aβ deposition compared to HC spheroids (FIG. 1J and FIG. 2B). In contrast to HC spheroid, however, ADAD spheroids showed a significant increase of Aβ deposition from PID22 to PID 28 (FIG. 2B). To confirm the extracellular deposition of Aβ in spheroid, cryo-sections of PID28 spheroids were immunostained using two different Aβ antibodies (6E10 and 82E1). Extracellular Aβ deposits were detected in ADAD spheroids by 6E10 or 82E1 antibodies, as observed in 5XFAD mouse brain sections (FIG. 1K and FIG. 2C). Transmission electron microscopy (TEM) with immunogold labeling against Aβ also showed gold signals in the extracellular domain in ADAD spheroids (FIG. 2E). Finally, to confirm that Aβ deposits observed in ADAD spheroids were generated through APP cleavage, the spheroids were treated with β-secretase inhibitor IV or DAPT from PID16 to 28, which led to the reduction of Aβ deposition (FIG. 2D). Taken together, these data demonstrate that ADAD 3D-CNs and spheroids contain elevated levels of extracellular Aβ deposition.

### Example 3. Tau Dysregulation in FAD 3D-CNs

In AD, tau protein becomes hyperphosphorylated, resulting in tau dissociating from microtubules and forming insoluble tau aggregates in the neurites and cell bodies. To investigate the level of phosphorylated tau (p-tau) in ADAD 3D-CNs compared to age-matched HC 3D-CNs, immunostaining analyses was carried out using AT8 (phosphor-Ser202/Thr205) and PHF1 (phosphor-Ser396/Ser404) p-tau antibodies. Both ADAD and HC neurites exhibited p-tau signals similar to the tau islands patterns seen in primary neurons (FIG. 3A and FIG. 4A). However, ADAD 3D-CNs displayed significantly higher levels of p-tau compared to HC 3D-CNs (FIG. 3A). ADAD 3D-CNs also showed higher signals for MC1 antibody, which detects conformationally misfolded tau, compared to HC 3D-CNs (FIG. 3B). In human AD brains, K63-linked ubiquitinated tau accumulates at high levels and is associated with enhanced tau seeding and propagation. By performing immunostaining using K63-linked ubiquitin and p-tau (PHF1) antibodies, it was found that ADAD 3D-CNs showed significantly higher volumes of K63-ubiquitin in PHF1-positive neurites compared to HC 3D-CNs (FIG. 3C).

Importantly, “beading” or “blebbing” in neurites is a morphological sign of dystrophic neurites in AD brain. Notably, spherical, beaded neurites were detected in ADAD 3D-CNs and spheroids that are positive for p-tau/K63-ubiquitin staining (FIG. 3C and FIG. 4B, see by arrows). The presence of beaded dystrophic neurites in ADAD neurons was confirmed by high-resolution TEM (FIG. 3D).

To test whether ADAD 3D-CNs would contain seed-competent tau that facilitates spreading of pathogenic tau, Fluorescence Resonance Energy Transfer (FRET) assay was carried out by transducing tau FRET reporters composed of tau P301S-Ruby2 and -Clover reporters, which dimerize and transmit FRET in the presence of seed-competent tau species. As shown in FIG. 3E, ADAD 3D-CNs exhibited significantly increased FRET signals compared to HCs, suggesting that ADAD 3D-CNs contain seed-competent tau. Intriguingly, the FRET signal in ADAD 3D-CNs was specifically located in beaded dystrophic neurite regions (FIG. 3E). Methanol fixation has previously been used to remove soluble proteins, including tau. Immunostaining using PHF1 p-tau antibody in methanol fixed CNs indicated that ADAD CNs exhibited elevated amounts of insoluble p-tau (FIG. 3F).

Moreover, GT-38 tau antibody which selectively labels the AD-specific tau strain were also used, and its abundance correlated with disease stage. Immunostaining of ADAD 3D-CNs showed GT-38-labeled tau albeit with low occurrences (FIG. 4C). Finally, whether Aβ deposits would underlie tau dysregulation in patient-derived neurons was tested by treating ADAD 3D-CNs with β-secretase inhibitor IV or γ-secretase inhibitor (DAPT) to prevent APP processing. It was found that inhibiting APP led to significant reduction of p-tau (AT8) and K63-ubiquitin+p-tau (PHF1) levels in ADAD 3D-CNs (FIG. 3G). Altogether, these results indicate that ADAD neurons manifest tau pathology that is dependent on the formation of Aβ deposits.

### Example 4. ADAD 3D-CNs and Spheroids Undergo Spontaneous Neurodegeneration

AD is a neurodegenerative disorder characterized by the loss of neurons, particularly in the hippocampus and cerebral cortex. To test if directly reprogrammed ADAD 3D-CNs would undergo spontaneous neurodegeneration, cell death in 3D-CNs was examined at multiple time points during reprogramming by Sytox-Green assay, a general cell death indicator. While both ADAD and HC reprogramming cells showed low levels of cell death prior to PID20, significantly higher levels of cell death were detected in ADAD 3D-CNs at PID30 and PID35, compared to HCs at same PIDs (FIG. 5A). These time points correspond to when neuronal identity is established during the conversion process (FIG. 1B and FIG. 1N). Neuronal death in spheroids was also examined by carrying out TUNEL staining on sections of spheroids at PID28. Consistent with 3D-CNs, ADAD spheroids showed significantly higher cell death levels compared to HC spheroids (FIG. 5B). The specificity of the neuronal death phenotype was further tested by culturing ADAD spheroids (labeled by turboRFP) immediately adjacent to HC spheroids (labeled by cGFP). Remarkably, ADAD spheroids demonstrated stark shrinkage in size by 50% from PID 17 to PID33, while HC spheroids exhibited only a 10% reduction during the same time interval (FIG. 5C right). In contrast, two adjacent HC spheroids derived from independent control donors did not undergo significant shrinkage from PID17 to PID33 (FIG. 5C left) indicating that AD-associated neurodegeneration was specifically recapitulated in ADAD spheroid. Since AD is also characterized by progressive degeneration of neurites, we examined neurite outgrowth in ADAD and HC spheroids at PID22 and PID28. HC spheroids maintained consistent neurite length from PID22 to PID28, while ADAD spheroids showed significant retraction of neurite outgrowth at PID28 (FIG. 5D). Lastly, it was tested if the neuronal death would be a phenotype downstream of Aβ deposits. Treating ADAD spheroids with β- or γ-secretase inhibitors led to significant reduction of neuronal death (FIG. 5E). Altogether, these results demonstrate that ADAD 3D-CNs and spheroids undergo spontaneous, AB-dependent neurodegeneration.

### Example 5. LOAD 3D-CNs and Spheroids Display Increased Aβ Deposition, Tau Dysregulation, Dystrophic Neurites, and Neuronal Cell Death

While reprogrammed ADAD neurons capture the adult-onset neuropathology resulting from disease-causing genetic mutations, it is not clear if LOAD samples would also manifest AD-related neuropathology. As such, it was tested if the 3D neuronal reprogramming could be applied to LOAD samples to model AD-associated neurodegeneration. It is noteworthy that Aβ deposition is not limited to AD patients but can be also present in non-demented elderly individuals. Thus, whether Aβ deposition in 3D-CNs from healthy individuals at different ages was first examined. While Aβ deposits were barely detected in the 3D-CNs derived from a young adult (22 years of age), Aβ deposition became more detectable with increasing ages (FIG. 6A). Therefore, for comparing Aβ between LOAD and HC CNs, it is critical to match the donor ages. Both LOAD and HC 3D-CNs displayed extracellular Aβ deposits detected by 6E10 and 82E1 Aβ antibodies (FIG. 7A and FIG. 6B); however, LOAD 3D-CNs (6 independent patients, 60-89 years old) showed higher levels of Aβ deposition compared to HC 3D-CNs from six independent controls of similar age range (FIG. 7A). Similarly, LOAD spheroids from six independent patients exhibited significantly higher levels of extracellular Aβ deposits at PID28 compared to age-matched HC spheroids from six independent control individuals (FIG. 7B and FIG. 6C). These data demonstrate the feasibility of recapitulating Aβ deposition in LOAD and aged HC neurons via 3D neuronal reprogramming.

In AD, tau tangles contain a mixture of 3R- and 4R-tau isoforms to adopt an AD-characteristic topology. Thus, endogenous expression of both 3R and 4R tau isoforms is a critical criterion when using reprogrammed human neurons for modeling tau pathology in AD. To examine if neurons reprogrammed from LOAD patients express both 3R and 4R tau isoforms, semi-quantitative PCR was carried out. Both 3R- and 4R-tau isoforms were detected with no overt difference in the 3R to 4R ratio between LOAD and HC 3D-CNs (FIG. 7C). Several lines of evidence suggest that increased tau phosphorylation is a reflection of brain aging in general. Interestingly, it was noted that the trend of increased tau phosphorylation is correlated with the increasing ages of control donors (FIG. 8A).

Phosphorylated tau (AT8) was detected in both LOAD 3D-CNs and aged-matched HC 3D-CNs when reprogrammed cells gained the neuronal identity at PID30 and PID35, but not at PID10 and 20 (FIG. 8B). Importantly in comparison between LOAD and HC, it was found that LOAD 3D-CNs exhibited significantly higher p-tau levels compared to HC 3D-CNs, as assessed by immunostaining with AT8 and PHF1 antibodies (FIGS. 7D and 8C). LOAD 3D-CNs also showed significantly higher signals for MC1 tau antibody which recognizes conformationally misfolded tau (FIG. 8D). Additionally, in LOAD 3D-CNs there was significant elevation of K63-ubiquitin+pathogenic tau signals, primarily located in beaded neurites, compared to HC 3D-CNs (FIG. 7E). Similar to ADAD 3D-CNs (FIG. 3E), seed-competent tau was also detected in LOAD 3D-CNs as measured by FRET signals, especially in the beaded neurite regions (FIG. 7F). Altogether, these results indicate that LOAD patient-derived neurons are capable of capturing tau dysregulation through 3D reprogramming.

Brain atrophy due to neuronal loss is a pathologic feature in LOAD patients. While LOAD and HC reprogramming cells did not display apparent cell death during the early phase of reprogramming (PID20), both PID30 and 35 time points showed significantly higher levels of Sytox signals in LOAD 3D-CNs compared to HC 3D-CNs (FIG. 9A and FIG. 9B). Live cell tracking over time confirmed the severe neuronal loss in LOAD 3D-CNs compared with HC 3D-CNs at later stages of reprogramming when the neuronal identity was evoked (FIG. 9C and FIG. 9D). LOAD spheroids also presented a significant increase of cell death, as revealed by TUNEL staining (FIG. 7G), and disintegration of neurites over time (FIG. 7H), in comparison to HC spheroid. Finally, culturing RFP-labeled LOAD spheroids adjacent to GFP-labeled HC spheroids revealed a ˜50% reduction of the size of LOAD spheroids from PID17 to PID33, while only ˜15-20% reduction of size was observed in the HC counterparts over the same period (FIG. 71). Taken together, these data demonstrate that LOAD 3D-CNs and spheroids recapitulate AD-associated neuropathology characterized by increased Aβ deposition, tau dysregulation, and neuronal death compared to age- and sex-matched HCs.

### Example 6. Effect of Inhibiting APP Processing in LOAD 3D-CNs and Spheroids on AD Neuropathology

LOAD 3D-CNs and spheroids represent a previously unavailable patient neuron-based system that endogenously captures AD-associated neuropathology. The interplay between Aβ deposition, tauopathy and neurodegeneration was tested by examining effects of reducing Aβ deposition on tau and neurodegeneration. This was assessed at two time points of reprogramming. First, treating reprogramming cells with β- or γ-secretase inhibitors at PID16 was started, a time point before the observed onset of Aβ deposition (FIG. 6D). Treating LOAD spheroids with β- or γ-secretase inhibitors before the formation of Aβ deposits significantly reduced Aβ deposits in the spheroids by 80-90% and cleaved Aβ species in the media compared to DMSO (FIG. 10A and FIG. 6E). Second, reprogramming cells were treated with β- or γ-secretase inhibitors at PID22, when moderate amount of Aβ deposits was already present (FIG. 6D). Inhibiting APP processing after Aβ deposition has already begun, resulted in either a non-significant effect (by β-secretase inhibitor IV), or only mild reduction (˜30% by DAPT) on Aβ deposition (FIG. 10A). Next, it was found that inhibition of Aβ formation before accumulation of Aβ deposits significantly decreased p-tau level and K63-ubiquitin/p-tau colocalization, whereas inhibition of Aβ formation at the later time point resulted in only non-significant effects on tau dysregulation (FIG. 10B). Lastly, neuronal death in spheroids treated with β- or γ-secretase inhibitors was examined. Similar to the effects on tau pathology, neuronal death in LOAD spheroids was reduced by nearly 50% when APP processing was inhibited starting at an early phase of reprogramming (PID16). However, secretase inhibitors had only minor effects on neuronal death if applied after the onset of Aβ aggregation (FIG. 10C). Together, these results demonstrate that the timing of blocking Aβ production is a crucial factor for the inhibition of Aβ formation and subsequent tauopathy and neuronal death in the LOAD 3D-CN and spheroids models.

### Example 7. LOAD Spheroids Exhibit Distinct Transcriptomes and Inhibition of Retrotransposable Element (RTE) Dysregulation Suppresses LOAD Pathologies

To deduce transcriptional patterns associated with LOAD, RNA sequencing (RNA-seq) was conducted on PID25 spheroids reprogrammed from five independent LOAD fibroblast lines and five sex- and age-matched HC fibroblast lines. Principle component analysis indicated a clear separation of samples based on disease status (HC vs LOAD) (FIG. 11A). By comparing protein-coding gene expression in LOAD and HC spheroid, 846 differentially expressed genes (DEGs) (P<0.05, |log 2FC|>0.58) were identified (FIG. 12A). Notably, several MMP (M10 matrix metallopeptidases) genes, including MMP1, MMP3 and MMP8 were among the most upregulated genes. MMPs are known to be linked to extracellular protein degradation including Aβ49, while MMPs upregulated in AD may also confer neuroinflammation, synaptic dysfunction, and neuronal death. Notably, gene ontology analysis revealed that upregulated DEGs in LOAD spheroids were significantly enriched for pathways associated with immune response and inflammatory responses, while downregulated DEGs were associated with nucleosome assembly, telomere organization, and memory (FIG. 12B).

While inflammation can result from various factors, it has been shown that activation of retrotransposon elements (RTEs) in aging and late-onset diseases may trigger cell-intrinsic inflammation. Suppression of RTE ameliorates age-associated inflammation and reduces tau activation and tau-induced neurotoxicity in tau transgenic Drosophila. Consequently, it was examined whether reprogrammed spheroids exhibited age-dependent changes in the expression of transposon elements (TEs). TE analysis from RNA-seq datasets of HC spheroids divided by age (36-60 years vs. 66-90 years) revealed differentially expressed TEs (DETEs) between age groups, with TEs linked to the older age group also enriched in LOAD spheroids (FIG. 12C). To determine whether inhibiting RTE expression could influence AD-associated phenotypes, LOAD spheroids and 3D-CNs were treated with the reverse transcriptase inhibitor lamivudine (3TC). Remarkably, 3TC treatment from PID16 to PID28 significantly reduced neuronal cell death in LOAD spheroids compared to the control H2O treatment (FIG. 12D). Additionally, 3TC considerably decreased Aβ formation and tau dysregulation, as evidenced by reduced Aβ deposition, p-tau, and K63-linked ubiquitin-positive tau signals (FIGS. 12E, 12F and 12G). This protective effect was not due to the loss of neuronal identity with 3TC treatment, as assessed by qPCR (FIG. 11B). These results demonstrate that perturbing RTE synthesis conferred protective effects in LOAD neurons and highlight the overall potential of using the patient-specific LOAD neurons to track changes in AD phenotypes for chemical interventions.

### Example 8: Discussion

These findings highlight the significant advance in modelling AD through 3D neuronal reprogramming: 1) the age-maintained patient-derived neurons recapitulate hallmark neuropathological features of AD, including extracellular Aβ deposition, tauopathy, and neurodegeneration, 2) the capacity to endogenously capture neuronal aspects of AD pathology without requiring additional cellular insults or transgenes, and 3) the potential to model late-onset neuropathology of LOAD. The finding that 3D-reprogrammed neurons derived from LOAD patients, without genetic mutations linked to ADAD, exhibit neurodegeneration-related phenotypes underscores the efficacy of 3D neuronal reprogramming in capturing AD-associated traits resulting from the donor's genetic background and epigenetic signatures. Although the current study provides evidence that inhibiting APP processing or dampening RTE promoted the survival of reprogrammed LOAD neurons, identifying other factors contributing to the late-onset degeneration of LOAD neurons would help inform strategies to slow neurodegeneration. Consequently, future research using whole genome-sequencing and chromatin profiling would facilitate the identification of genetic and epigenetic changes underlying the neurodegeneration phenotype observed in patient-specific neurons. The 3D neuronal reprogramming could serve as a platform to investigate genetic risk factors that may function in neurons, contributing to individual variability and vulnerability to neuropathology of AD. It is also noteworthy that Aβ deposits and p-tau signals correlate with aging even in neurons derived from healthy individuals (FIG. 6A and FIG. 8A). This observation emphasizes the potential of using 3D neuronal reprogramming to explore how neuronal aging generally increases susceptibility to late-onset neurodegeneration.

Here, the significant differences of 3D direct reprogramming modeling approach provided in this study compared with animal and human cellular models based on ADAD-associated mutations or 2D direct reprogramming approaches is highlighted. While mouse models based on overexpressing gene mutations associated with ADAD offer valuable insights into Aβ plaque formation, they show limited tau pathology. It typically requires additional expression of human tau harboring mutations associated with primary tauopathy or seeding mutant human APP knock-in mouse brain with human AD brain-derived tau to develop the tau pathology. As for human neurons, previous studies focused on overexpressing APP and Presenilin containing ADAD mutations in human stem cell lines, in which they provided evidence that these cell lines developed Aβ accumulation and tauopathy.

Generation of age-equivalent, AD patient-derived neurons in 2D-culture environment has been recently carried out by using Ngn2/Asc11-based direct conversion approach, which revealed AD patient-derived neurons exhibited age-dependent instability of mature neuronal fate, increased post-mitotic senescence and pro-inflammatory signature. The current disclosure focuses on the feasibility of endogenously capturing critical neuropathological features of AD in patient-derived neurons by developing 3D-direct reprogramming approaches. In this 3D culture system, AD patient-derived neurons not only exhibited robust extracellular Aβ deposition and tau dysregulation, but also developed severe neurodegeneration. Moreover, this study shows validation that inhibiting APP processing in patient-derived neurons and spheroids alleviates tau pathology, and importantly, it also reduces the neurodegeneration phenotype. However, this effect was only minimal when APP processing was inhibited after LOAD neurons had already started forming Aβ deposits. Therefore, AD modelling through 3D neuronal conversion can serve as a platform to study the interplay between Aβ deposition, tau dysregulation and neurodegeneration, and identify gene targets or small molecules, as exemplified by 3TC experiments, that can perturb the disease progression.

Results in this study also demonstrate the sufficiency of directly reprogrammed patient neurons to capture key neuronal features of AD which prompts new avenues for studying how these phenotypes may change when AD neurons interact with other cell types in the context of aging and disease. In this regard, aging may impact glial cells such as astrocytes and microglia. Thus, robust direct reprogramming methods for generating aged astrocytes and microglia would enable the study of neuron-glia interactions during the aging process and age-associated disease progression. In conclusion, the current study demonstrates the effectiveness and sufficiency of 3D-cultured, patient-derived neurons through miRNAs-mediated direct reprograming for modeling critical neuropathological hallmarks of AD. This approach presents opportunities to investigate molecular events intrinsic to neurons that drive AD-associated neurodegeneration and may serve as a patient-specific neuron platform for testing various compounds or target genes for personalized therapeutic interventions.

### Example 9: Experimental Procedures

### Fibroblast Cell Lines

Adult dermal fibroblast cell lines from familial AD patients were obtained from University College London (UCL33, UCL53 and UCL803) and Coriell Institute for Medical Research (AG06840). Adult dermal fibroblast cell lines from biomarker positive late-onset AD patients were acquired from Washington University Knight Alzheimer's Disease Research Center (ADRC), which includes FA15-552, FA12-453, FA17-623 and FA18-633. Two additional skin fibroblast cell lines from late-onset Alzheimer's disease patients were purchased from Coriell (AG05810 and AG06869). The skin fibroblast cell lines derived from healthy control individuals were obtained from Washington University Knight ADRC (biomarker negative: FA18-634 and FA12-463) and Coriell (AG08260, AG04453, AG04060, AG05838, AG13246, AG11020, AG13369, AG08379, AG14251, AG04356, GM02171, AG07307 and AG11246). Detailed information of the list of fibroblast cell lines used in this study is provided in Table 1. All fibroblast cell lines were cultured in fibroblast medium (FM) containing 15% fetal bovine serum (FBS): Dulbecco's Modified Eagle Medium (DMEM, Invitrogen) with high glucose containing 15% FBS, 0.1% 55 mM beta-mercaptoethanol, 1% IM HEPES buffer, 1% nonessential amino acids, 1% sodium pyruvate, 1% GlutaMAX and 1% penicillin/streptomycin solution (all from Gibco). Cell culture medium was routinely checked to ensure there was no mycoplasma contamination. Cells were maintained up to 15 passages.

### Lentiviral Production:

Lentiviruses were generated as previously described. Briefly, the second-generation lentiviral system for production was used by transfecting the transfer plasmid, packaging plasmid (psPAX2) and envelope plasmid (pMd2G) in HEK293LE cells. 60-70 hours after transfection, the supernatant containing lentivirus was filtered through a 0.45 μm PES membrane and concentrated at 70,000 g for 2 h at 4° C. Viral pellets then were resuspended in PBS, aliquoted and stored at −80° C. until used for transduction (viruses can be stored in −80° C. for up to a year).

### 3D Direct Neuronal Reprogramming:

The 3D direct neuronal reprogramming protocols were established based on modifications of previously published 2D direct reprogramming protocol. Briefly, human fibroblasts were spin-infected (1000 g for 30 mins at 37° C.) with lentiviral cocktail containing rtTA, pTight-9-124-BclxL, MYT1L and NEUROD2. After 16-18 hours incubation at 37° C., the infected cells were washed once with PBS and fed with fresh fibroblast medium containing 10% FBS and 1 μg/ml doxycycline (Sigma, cat. #D9891). The fibroblast medium was changed every other day with 10% FBS, 1 μg/ml doxycycline and antibiotics. On day 7, cells were trypsinized, resuspended in transiting medium and counted for replating.

For thin gel culture, 0.1×10⁶ cells resuspended in 100 μL transiting medium containing 15% MATRIGEL™ (Corning, cat. #354230) were immediately transferred to each well of an optically clear 96-well plate (Perkin Elmer, cat. #6055302) to form a thin gel. The plates were incubated at 37° C. overnight to let the gel solidify and form a thin layer (˜100 μm) MATRIGEL™/cell mixture at the bottom of the well. The next day (day 8), 200 μL pre-warmed Neurobasal-A medium was added to the thin gel.

For spheroids culture, 0.5×10⁶ cells were centrifuged at 400 g for 2.5 min at room temperature to form a cell pellet. The cell pellet was transferred to the center of a transwell insert (Corning, cat. #353095) in a 24 well plate by using a wide-bore tip (200 μL, USA Scientific. cat. #1011-8410) to form a spheroid. 250 μL transiting medium was added to the bottom of the well and the plate containing spheroids was placed in 37° C. incubator overnight. On day 8, the spent transiting medium in the bottom well of transwell plates was replaced by 250 μL fresh Neurobasal-A medium. 100 μL of 50% MATRIGEL™ diluted in Neurobasal-A medium was added into the transwell insert. After incubation at 37° C. for 3 hours to overnight to let the MATRIGEL™ solidify and form a “MATRIGEL™ blanket”, 250 μL fresh Neurobasal-A medium was added on top of the “MATRIGEL™ blanket”.

After replating, half media changes (Neurobasal-A medium) were performed every other day for both thin gel culture and spheroids culture. On day 14 and after, Neurobasal-A medium was replaced by Brainphys neuronal medium for half media changes. Puromycin (3 μg/mL) was added to the medium from PID3 to PID14 and G418 (geneticin, 300 μg/ml) was added to the media at PID5-PID10 for selection. 1 μg/ml doxycycline was added until PID30.

Transiting medium: Neurobasal A medium mixed with fibroblast medium (10% FBS) at ratio of 1:1. No antibiotics were added to transiting medium.

Neurobasal-A medium: Neurobasal-A medium (Gibco, cat. #10888022) containing B-27 plus supplement (Gibco, cat. #A3582801), GlutaMAX (Gibco, cat. #35050061) and supplemented with doxycycline (1 μg/mL), valproic acid (1 mM), dibutyl cAMP (200 μM), BDNF (10 ng/mL), NT-3 (10 ng/ml), retinoic acid (1 μM), ascorbic acid (200 nM), and RVC (RevitaCell Supplement, 1x).

BrainPhys neuronal medium: BrainPhys neuronal medium (Stemcell Technologies, cat. #05790) containing NeuroCult SMIneuronal supplement (Stemcell Technologies, cat. #05711), N2-A supplement (Stemcell Technologies, cat. #07152) and following supplements: doxycycline (1 μg/mL), valproic acid (1 mM), dibutyl cAMP (200 μM), BDNF (10 ng/ml), NT-3 (10 ng/ml), Retinoic acid (1 μM), and ascorbic acid (200 nM).

### β-Secretase Inhibitor IV, DAPT and Lamivudine (3TC) Treatment

β-secretase inhibitor IV (cat #565788), DAPT (cat #D5942), and lamivudine (3TC) (cat #PHR1365) were purchased from Millipore Sigma. To make the stock solution, 5 mM β-secretase inhibitor IV and 40 mM DAPT were dissolved in DMSO and 100 mM 3TC was dissolved in sterile water. For thin gel culture, 2.5 μM β-secretase inhibitor IV, 20 μM DAPT, or 100 μM 3TC was added to the fresh media and used for half media changes starting at PID16 or PID22. For spheroids culture, 5 μM β-secretase inhibitor IV, 40 μM DAPT, or 100 μM 3TC was added to the fresh media and used for half media changes starting at PID16 or PID22.

### Fixation of 3D Cultures, OCT Embedding and Sectioning of Neuronal Spheroid

Fixation: For thin gel culture, 100 μL 4% (wt/vol) paraformaldehyde (PFA) solution was added to each well; For spheroids culture, 250 μL 4% (wt/vol) PFA solution was added in the bottom well and another 250 μL on the insert. After incubating overnight at room temperature, the PFA solution was removed, and the 3D cultures were washed with PBS once. Plates containing fixed 3D cultures were filled with PBS and sealed with parafilm to prevent from evaporation. The fixed 3D cultures can be stored at 4° C. for 6-12 months.

OCT embedding and sectioning of neuronal spheroid: Fixed neuronal spheroids were soaked in 30% sucrose (w/v) overnight at 4° C. Neuronal spheroids were manually detached from the transwell insert, embedded in OCT compound (Tissue Tek), and followed by snap-frozen in liquid nitrogen to form OCT blocks. The OCT embedded spheroids were cryo-sectioned at 20 μm and mounted on positively charged Superfrost slides (VWR). Slides can be stored at −80° C. until further use.

### Immunofluorescence Staining, 3D Reconstruction, and Quantification

Immunofluorescence staining of thin gel: The fixed thin gel cultures were permeabilized in PBST (0.2% (vol/vol) TritonX-100 in PBS) at room temperature for 1h and blocked with blocking buffer (5% (wt/vol) BSA, 2% (wt/vol) normal goat serum, and 0.2% (vol/vol) TritonX-100) overnight at 4° C. The blocking buffer was removed from thin gel culture plates and primary antibodies diluted in blocking buffer were added to the plate followed by incubation overnight at 4° C. with gentle rocking. After washing 5 times with PBST, the cells were then incubated with secondary antibodies diluted in blocking buffer at room temperature for 3-5 hours or 4° C. overnight. Cells were washed 5 times with PBST, incubated with DAPI (Sigma) for 20 min at room temperature if needed and sealed with a drop of anti-fade prolong gold reagent (Life technology). Plates were sealed with parafilm and stored at 4° C. before imaging. The immunofluorescence images were taken by Leica SP5X white light laser confocal system.

Immunofluorescence staining of sections from spheroid: Sections were permeabilized in PBST for 10 min followed by incubating in blocking buffer for 30 min at room temperature. The sections were incubated with primary antibodies in blocking buffer at 4° C. overnight, then washed three times with PBST. The sections were incubated with secondary antibodies in blocking buffer for 30-60 min at room temperature, followed by three washes with PBST. The sections were then stained with DAPI for 10 min at room temperature and mounted in anti-fade prolong gold reagent (Life technologies). The immunofluorescence images were taken by Leica SP5X white light laser confocal system.

Whole mount immunofluorescence staining of spheroid: For whole-mount immunostaining of neuronal spheroid, 3D cell culture clearing kit (abcam: ab243299) was used with modifications. Briefly, fixed spheroids on transwell inserts were permeabilized by sequential dehydration and rehydration using different concentrations of ethanol. Specifically, spheroids were sequentially soaked in 50% ethanol in PBS, 80% ethanol in PBS, 100% ethanol, 80% ethanol in DMSO, 80% ethanol in PBS, 50% ethanol in PBS, and PBS (5 min each, room temperature). Spheroids were incubated in the Tissue Clearing Penetration buffer (from the kit) for 30-60 mins at room temperature followed by blocking overnight in blocking buffer. Then the spheroids were incubated with primary antibodies diluted in blocking buffer at 4° C. for 24-48 hours with gentle rocking, followed by washing 5 times with PBST, 10 mins each. Fluorescent secondary antibodies diluted in blocking buffer were added to the spheroids and incubated overnight at 4° C. After 5 washes with PBST, the 3D Cell Culture Clearing Reagent (from the kit) was added to completely cover the spheroids and incubated for at least 1 hour at room temperature with gentle rocking. The spheroids should look transparent under light microscope. The spheroids mounted in this clearing reagent can be stored in 4° C. for at least 6 months with proper parafilm sealing of the plates and avoiding light. Immunofluorescence images were taken on an upright the Nikon A1RHD25 MP multi-photon microscope.

For the list all the antibodies and dilution ratios used in this study, please see Table 2.

TABLE 2
List of Antibodies

| | | | | IF for 3D- | |
|---|---|---|---|---|---|
| | | | | CNs or | Whole mount |
| | | | | sections from | IF staining for |
| Antibody | Host | Supplier | Cat # | spheroid | spheroid |
| TUBB3 | Rabbit | Biolegend | 802001 | 1:2500 | 1:1000 |
| TUBB3 | Mouse | Biolegend | 801202 | 1:1000 | 1:500 |
| TUBB3 | Chicken | Novus | NB100- | 1:1000 | 1:500 |
| | | | 1612 | | |
| NCAM1 | Mouse | Santa Cruz | SC-106 | 1:50 | |
| VGLUT1 | Mouse | Millipore | MAB5502 | 1:200 | |
| MAP2 | Rabbit | Cell signaling | #4542 | 1:200 | |
| NeuN | Mouse | Chemicon | MAB377 | 1:200 | |
| Amyloid-3 (6E10) | Mouse | Biolegend | 803001 | 1:200 | 1:100 |
| Amyloid-3 (82E1) | Mouse | IBL | 10323 | 1:200 | 1:100 |
| P-TAU (AT8) | Mouse | Invitrogen | mn1020 | 1:200 | |
| P-TAU (PHF1) | Mouse | Gift | N/A | 1:400 | |
| TAU (MC1) | Mouse | Gift | N/A | 1:400 | |
| K63-linked | Rabbit | Abcam | ab179434 | 1:200 | |
| UBIQUITIN | | | | | |
| TAU(GT38) | Mouse | Abcam | ab246808 | 1:200 | |

For quantification of the total volume of Aβ deposits, volume and area of the spheroid, co-localization analysis in tau related pathologies, and number of TUNEL positive cells or total cells, the “surface” module in Imaris was used for 3D reconstruction and quantification. For measuring the intensity of neurite outgrowth in spheroid, Image J was used by measuring the mean intensity at each region (distal, middle, and proximal).

### FRET Live Imaging:

Tau RD P301S Ruby2 and tau RD P301S clover plasmids were constructed as previously described. Lentiviral FM5-Ruby2 or FM5-Clover plasmid drives the tau repeat domain (RD, aa246 to 378) with the disease-associated P301S mutation under the human ubiquitin C promoter. The lentiviruses to express tau RD (P301S)-Ruby2 and RD (P301S)-clover were transduced into reprogrammed cortical neurons at PID19 using Polybrene (Sigma-Aldrich, H9268). The next day, the media containing lentiviruses were removed and fresh Brainphys neuronal medium was added to the cells. Live cell imaging was performed at PID25, PID27 and PID29 using Leica SP5X white light laser confocal system. To detect FRET, cells were illuminated with 488 nm light and emission was captured between 620 to 700 nm. To avoid false positive signal in the FRET channel, cells in separate wells were transduced with single reporter (RD (P301S)-Ruby2 or RD (P301S)-clover only) to set imaging thresholds.

PCR Analysis of 3-Repeat (3R) and 4-Repeat (4R) Tau mRNA Levels

Semi-quantitative PCR for analyzing 3R and 4R mRNA expression was carried out as previously described. Briefly, cDNAs that were reverse transcribed from RNAs of LOAD and HC 3D-CNs were used for PCR amplification using suitable primers flanking exon 10 of tau. The PCR products were run on a 2% agarose gel and images were taken by Chemidoc MP imaging system (BIO-RAD). Image J was used to analyze the pixel intensity of 3R (288 bp) and 4R (381 bp) bands.

### Electrochemiluminescence Assay for Aβ42 Detection

For Aβ42 detection, V-PLEX Plus Aβ Peptide Panel 1 (6E10) Kit (Meso Scale Discovery (MSD), K15200E0) was used as previously described. 2-3 neuronal spheroids from the same cell line were pooled together and lysed in RIPA buffer using a sonicator. Spheroids reprogrammed from 4 FAD fibroblast lines and 4 age- and sex-matched healthy control lines were used for the experiment. Aβ42 levels were measured using the MESO QuickPlex SQ 120 (multiplexing imager, MSD) following the manufacturer's instructions. Aβ42 level was normalized to the total protein level in each sample.

RNA Preparations and Quantitative PCR (qPCR)

Total RNA was extracted from thin gel cultures or neuronal spheroids using TRIZOL™ (invitrogen) followed by RNeasy® Micro Kit (Qiagen, Cat #74004). Briefly, cells in thin gel or spheroids were lysed by TRIZOL™ at room temperature for 5 mins. 0.2 mL of chloroform was added per 1 mL of TRIZOL™ reagent and incubated for 2-3 mins at room temperature. The TRIZOL™/chloroform mixtures were centrifuged for 5 mins at 12,000g at 4° C. Aqueous phase was transferred to a fresh microcentrifuge tube and mixed with 1 volume of 70% ethanol. Up to 700 μL of clear lysates/70% ethanol mixture was applied to RNeasy MinElute column. Washing and elution were performed following the instructions from the RNeasy Micro Kit

Reverse transcription was performed using SUPERSCRIPT IV™ First Strand Synthesis SuperMix (Invitrogen, 18090050) according to the manufacturer's protocol. qPCR was performed with SYBR Green PCR Master Mix (Applied Biosystems, 4309155) using the STEPONE PLUS™ Real-Time PCR System (Aβ Applied Biosystems, Germany). All samples were run with two technical replicates. All absolute data were normalized to GAPDH and fold change was calculated based on the 2-4ACT method. The sequences of primers used for qRT-PCR are as listed in Table 3.

TABLE 3
List of Primer sequences

| Gene | |
|---|---|
| name: | DNA Sequence (5'-3') |
| MAP2 | Forward: CCCTTTGAGAACACGACACA |
| (Human) | (SEQ ID NO: 10) |
| | Reverse: TCTGTTAGCGGTGCTGAGGT |
| | (SEQ ID NO: 11) |
| MAPT | Forward: AAGTCGCCGTCTTCCGCCAAG |
| (Human) | (SEQ ID NO: 12) |
| | Reverse: GTCCAGGGACCCAATCTTCGA |
| | (SEQ ID NO: 13) |
| VGLUT1 | Forward: ACGTGCGCAAGTTGATGA |
| (Human) | (SEQ ID NO: 14) |
| | Reverse: TCACGTTGAACCCAGAGATG |
| | (SEQ ID NO: 15) |
| SNAP25 | Forward: TCGTGTAGTGGACGAACGG |
| (Human) | (SEQ ID NO: 16) |
| | Reverse: TCTCATTGCCCATATCCAGGG |
| | (SEQ ID NO: 17) |
| ACTL6B | Forward: CTTCCACATCGACACCAATG |
| (Human) | (SEQ ID NO: 18) |
| | Reverse: TTTGGCTCAGACTTGACGTG |
| | (SEQ ID NO: 19) |
| TBR1 | Forward: ACAATTTTCCTGACTCCAAGGAC |
| (Human) | (SEQ ID NO: 20) |
| | Reverse: ACTGTGACGAAGCTCAGAGAC |
| | (SEQ ID NO: 21) |
| POU3F2 | Forward: GCTGTGGGAGAGAGAGGAGA |
| (Human) | (SEQ ID NO: 22) |
| | Reverse: GCAGGCTGTAGTGGTTAGACG |
| | (SEQ ID NO: 23) |
| GAPDH | Forward: TTGAGGTCAATGAAGGGGTC |
| (Human) | (SEQ ID NO: 24) |
| | Reverse: GAAGGTGAAGGTCGGAGTCA |
| | (SEQ ID NO: 25) |

### Electrophysiology

Whole-cell patch-clamp recordings were performed as previously reported 29, 66, 67. Briefly, reprogrammed cortical neurons were transduced with pSynapsin-RFP at PID3 to label neurons. At PID 14, human astrocytes (Sciencell, cat. #1800) were added on top of the neurons for co-culturing.

Brainphys neuronal medium and Astrocyte medium (ScienCell, cat. #1801) were mixed at 1:1 for half media changes at PID14 and after. Whole cell patch clamp was performed at PID30. During recording, reprogrammed cortical neurons were transferred to a recording chamber with continuous perfusion (2 ml/min, 32° C.) of oxygenated, regular aCSF (in mM: 125 NaCl, 25 glucose, 25 NaHCO₃, 2.5 KCl, 1.25 NaH₂PO₄, 2.5 CaCl₂), 1.2 MgCl₂ equilibrated with 95% oxygen, 5% CO2 plus 2.5 CaCl₂), 1.2 MgCl2; 310 mOsm). Individual neurons were visualized and identified by IR-DIC microscopy (Nikon FNI microscope and Photometrics Prime camera). Borosilicate glass pipettes (World Precision Instruments, Inc) with open tip resistance of 3-7 MQ2 were used for whole-cell recording. Pipettes were filled with potassium gluconate solution containing the following (in mM: 120 K-Gluconate, 10 KCl, 2 EGTA, 10 HEPES, 2 MgATP, and 0.3 Na2GTP; pH 7.25 with KOH; 280-290 mOsm). Recordings were acquired using pCLAMP 10.4 software with a MultiClamp 700B amplifier and DIGIDATA® 1550 digitizer (Molecular Devices). Action potentials were elicited by current injection with 8 pA increments in current-clamp mode. From voltage-clamp mode, voltages step (100 ms) in increments of 10 mV were applied from a holding potential of −70 mV to monitor currents. Data were acquired at 5 kHz sampling rate and filtered at 2 kHz.

### SYTOX™ Assay and TUNEL Assay

SYTOX™: for thin gel cultured cortical neurons, 0.1 μM SYTOX™ green nucleic acid stain (Invitrogen, S7020) and 1 μg/mL of Hoechst 33342 (ThermoFisher Scientific, H3570) were added into cell media. Samples were incubated for at least 30 mins at 37° C. prior to the live cell imaging. Images were captured with a GE InCell 200 fluorescence microscope and analyzed by InCell investigator and developer image analyses software. Quantifications were performed by counting the percentage of SYTOX™ positive cells over Hoechst.

TUNEL assay: DeadEND Fluorometric TUNEL system kit was purchased from Promega (G3250) and TUNEL assay was performed according to the manufacturer's description. Briefly, the cryosections of neuronal spheroids were permeabilized by 0.2% TritonX-100 in PBS for 5 mins followed by two 5 mins washes in PBS. The sections then were equilibrated in equilibration buffer for 5-10 mins at room temperature and labeled by TdT reaction mix for 1 hour at 37° C. in a humidified chamber. The sections were immersed in 2×SSC for 15 min to stop the reaction, followed by three 5 mins washes in PBS. The sections then were counterstained with DAPI and mounted in anti-fade prolong gold reagent (Life technologies). Immunofluorescence images were taken by Leica SP5X white light laser confocal system.

### Electron Microscopy

High-pressure freezing: Reprogrammed cortical neurons were seeded and grown on 3 mm sapphire discs (Leica) until PID30. The 100 μm cavity of A-type specimen carrier (Leica) was filled with 20% BSA in culture medium and sapphire discs with cells facing the cryoprotectant was transferred on top of the carrier. A 200 μm thick ring for alternative spacing was placed on top of the assembly that was then vitrified using high-pressure freezing machine (Leica EM ICE).

Neuronal spheroids (PID28) were fixed for one hour in 4% paraformaldehyde in PBS, rinsed three times in PBS and then embedded in 4% agarose in PBS. Embedded specimens were cut into 200 μm sections using a vibratome (Leica VT1200S). Sections were placed in a 300 μm cavity of a B-type specimen carrier (Leica) filled with 20% BSA in PBS. The assembly was covered with a flat side of another B-type carrier and specimens were vitrified using a high-pressure freezing machine (Leica EM ICE).

### Freeze-Substitution

The frozen specimens were stored in liquid nitrogen until further processing. The freeze-substitution process was performed using a cocktail of 0.1% uranyl acetate in acetone and EM AFS2 machine with FSP robot for automated reagent handling (Leica). Briefly, samples were kept for 50 hours at −85° C. and then warmed up over a period of 11 hours to −50° C. At −50° C. samples were washed four times in ethanol for 30 minutes each before gradual infiltration with HM20 resin (Electron Microscopy Sciences). Polymerization of the resin was done with a UV light source and was carried out at −50° C. for 48 hours followed by post-polymerization at room temperature for 2 days.

### Immunogold Labelling

Thin sections of 70-80 nm were cut with an ultramicrotome (UC7, Leica) and placed on nickel grids (Ted Pella). Blocking was performed with 1% BSA in PBS and sections were incubated with primary antibody (Aβ antibody, 6E10) prepared at 1:20 dilution in the blocking buffer overnight at 4° C. Grids were washed five times for five minutes each in the blocking buffer. Following this, sections were incubated with 12 nm gold-conjugated donkey anti-mouse (Jackson ImmunoResearch Labs) secondary antibody (1:30 dilution in the blocking buffer) for 1 hour at room temperature. Sections were then washed in PBS and ultrapure water, dried and imaged on a TEM (Jeol JEM-1400 Plus) at 120 kV.TEM images were acquired using an AMT Nanosprint 15-MkII sCMOS camera.

### RNA-Seq Cell Collection, Sequencing, Data Processing, and Analysis

For comparing the transcriptome between LOAD vs HC spheroid, 3 biological replicates from 5 independent cell lines per disease group were used for RNA submission. Each replicate consisted of RNAs pooled from 2-3 spheroid. For RNA-seq of thin gel samples harvested at different time points, 2 biological replicates from one control line and one LOAD line were used for RNA-seq. Each replicate contained RNAs pooled from 5-10 wells of a 96 well plate. RNA samples that passed quality control were submitted to Genome Access Technology Center at Washington University for library preparation and sequencing. NovaSeq6000 using SMARTer 150PE with 30 million reads per sample input was used for sequencing. FastQC was used to determine sequencing quality and identify adapter contamination. Raw fastq files were trimmed using Cutadapt v3.2 for adapter contamination and low sequencing quality. Trimmed sequences were mapped to GRCh38 genome reference using STAR v2.7.10a. Raw gene counts were extracted using STAR option--quantMode GeneCounts. Differential gene expression analysis was performed using RUVSeq and DESeq2 normalizing to sequencing depth and removing unwanted variation.

### Transposable Elements (TE) Analysis

Trimmed reads from RNA-seq data analysis aligned to GRCh38 human genome using the STAR v2.7.10a with the additional parameters as recommended in the TE transcripts manual: -winAnchorMultimapNmax 100 and -outFilterMultimapNmax 100. TE transcript counts were generated based on RepeatMasker database using TEtranscripts v2.2.373. Differentially expressed TEs at the subfamily level between two different conditions were identified by DESeq2.

### Mass Spectrometry Analyses of Aβ

Aβ38, 40, 42 were quantified by mass spectrometry as previously described with some modifications. 0.5-1 mL of spheroids media was immunoprecipitated with monoclonal anti-Aβ mid-domain antibody (HJ5.1, anti-Aβ13-28) conjugated to Sepharose beads (30 μL, 3 mg/mL). Aβ was digested on beads with 50 μL of LysN (0.25 ng/μL) in 25 mM triethyl ammonium bicarbonate (TEABC). Digests were desalted by C18 TopTip (Glygen). Before eluting samples, 3% hydrogen peroxide and 3% formic acid (FA) in water was added onto the beads, followed by overnight incubation at 4° C. to oxidize the peptides containing methionine. The cluent was lyophilized and resuspended in 25 μL of 2-10% FA and 2-10% acetonitrile and 20 nM BSA digest prior to mass spectrometry analysis on nanoAcquity UPLC system (Waters) coupled to Orbitrap Fusion mass spectrometer (Thermo Scientific) operating in selected reaction mode. Concentrations of each peptide were estimated using internal standards containing Aβ38, 40, 42 uniformly labeled with 15N (0.01875, 0.125 and 0.0125 ng/μL, respectively. rPeptide).

### Statistics

For quantifying immunostaining data from thin gel culture, 3 to 4 confocal images taken from different area of each well for 3 wells of cell culture per each cell line, with 3 to 6 independent cell lines per disease group (AD vs HC) being used for analysis in each experiment. For quantifying whole mount immunostaining data in spheroids culture, 1 confocal image taken from the whole area of a spheroid, 2-3 spheroids per cell line, and 3 to 6 independent cell lines per disease group were used for analysis. For quantifying TUNEL data using spheroids sections, 3 to 4 confocal images taken from different area in each section for 3 sections covering different planes of a spheroid, with 2-3 spheroids of cells per cell line, and 3 to 6 independent cell lines per disease group were used for analysis. For harvesting RNA and qPCR analysis, 2-3 spheroids per cell line were pooled together for harvesting one RNA sample, 2-3 RNA samples per cell line, and at least 3 independent cell lines per disease group or per treatment were used for the experiments. An extra technical duplicate for each sample was used for qPCR analysis. Statistical analyses were performed in GraphPad Prism (version 9.5.1 (528)). Specific “n” information and statistical analysis method for each assay can be found in the figure legend. Generally, two-tailed paired or unpaired Student's t-tests were performed for datasets containing two groups. One-way or two-way ANOVA analyses were used for datasets containing more than two groups.

What is claimed is:

1. A method of generating a reprogrammed cortical neuron or neuronal spheroids from a patient-derived somatic cell, comprising:
a. providing the patient-derived somatic cell, wherein the patient has been diagnosed with or is suspected of having Alzheimer's disease (AD);
b. expressing miR-9/9*, miR-124, and at least one exogenous transcription factor, in the patient-derived somatic cell; and
c. culturing the patient-derived somatic cell in a 3D-culture, thereby reprogramming the somatic cell to a reprogrammed cortical neuron or neuronal spheroid.

2. The method of claim 1, wherein the at least one exogenous transcription factor comprises NEUROD2, ASCL1 or MYT1L.

3. The method of claim 2, wherein the at least one exogenous transcription factor comprises NEUROD2 and MYT1L.

4. The method of claim 1, wherein the method does not comprise induction of pluripotency in the somatic cell.

5. The method of claim 1, wherein the patient-derived somatic cell is an adult human fibroblast of mesodermal origin.

6. The method of claim 1, wherein the patient has been diagnosed with or is suspected of having Late Onset Alzheimer's disease (LOAD).

7. The method of claim 1, wherein the patient is at least 60 years or more of age.

8. The method of claim 1, wherein the reprogrammed cortical neuron or neuronal spheroids comprises one or more age signatures of the patient-derived somatic cell, selected from epigenetic clock, telomere length, and transcriptomic changes.

9. The method of claim 1, wherein the reprogrammed cortical neuron or neuronal spheroids exhibits one or more age-related LOAD neuropathology of the patient.

10. The method of claim 9, wherein the one or more age-related LOAD neuropathology comprises extracellular Aβ deposition, dystrophic neurites, hyperphosphorylated, K63-ubiquitin-positive tau, seed-competent tau, spontaneous neuronal death, increased levels of Aβ42, active GSK-3β, phosphorylated tau, endosomal abnormalities, oxidative stress, or any combination thereof.

11. The method of claim 1, wherein the miRNA, or the at least one exogenous transcription factor, or both are encoded by a nucleic acid sequence contained in a vector.

12. The method of claim 11, wherein the vector further comprises one or more regulatory elements comprising a promoter, an enhancer, a UTR, a termination sequence, an IRES or any combination thereof, operably linked to the nucleic acid sequence encoding the miRNA or at least one exogenous transcription factor.

13. The method of claim 12, wherein the promoter is an inducible promoter.

14. The method of claim 11, wherein the vector is a viral vector selected from adenovirus, adeno-associated virus, a retrograde virus, retrovirus, herpesvirus, lentivirus, poxvirus or papilloma virus expression vector.

15. The method of claim 11, wherein the nucleic acid sequence comprises a sequence set forth in SEQ ID NO: 1 and SEQ ID NO: 4, or a sequence at least about 80% identical thereto.

16. The method of claim 11, wherein the nucleic acid sequence comprises a sequence set forth in any one or more of SEQ ID NO: 7, SEQ ID NO: 8 and SEQ ID NO: 9, or a sequence at least about 80% identical thereto.

17. A method of screening for a drug for treatment of Alzheimer's disease (AD), comprising:
a. providing a patient-derived somatic cell;
b. expressing miRNA-9/9*, miRNA-124 and at least one exogenous transcription factor in the patient-derived somatic cell; and
c. culturing the patient-derived somatic cell in a 3D-culture, thereby reprogramming the somatic cell to a reprogrammed cortical neuron cell or neuronal spheroid;
d. contacting the reprogrammed cortical neuron cell or neuronal spheroids with the drug; and
e. following step (d), analyzing the treated reprogrammed cortical neuron cell or neuronal spheroids for a level of at least one AD-related neuropathology; wherein a reduction in the level of the at least one AD-related neuropathology in the reprogrammed cortical neuron cell or neuronal spheroids in comparison to a control spheroids is indicative of the effectiveness of the drug for treating Alzheimer's disease.

18. The method of claim 17, wherein the AD is a late-onset AD (LOAD).

19. The method of claim 17, wherein the at least one AD-related neuropathology comprises extracellular Aβ deposition, dystrophic neurites, hyperphosphorylated, K63-ubiquitin-positive tau, seed-competent tau, spontaneous neuronal death, increased levels of Aβ42, active GSK-3β, phosphorylated tau, endosomal abnormalities, oxidative stress, or any combination thereof.

20. A reprogrammed cortical neuron cell or neuronal spheroid, generated by a method comprising:
a. providing a patient-derived somatic cell;
b. exogenously expressing miRNA-9/9*, miRNA-124, and at least one transcription factor in the patient-derived somatic cell; and
c. culturing the patient-derived somatic cell in a 3D-culture, thereby reprogramming the somatic cell to the reprogrammed cortical neuron cell or neuronal spheroid;
wherein the reprogrammed cortical neuron or neuronal spheroids exhibits one or more age-related phenotype(s) of the patient-derived somatic cell.

21. The reprogrammed cortical neuron or neuronal spheroids of claim 20, wherein the one or more age-related phenotype(s) is epigenetic clock, telomere length, and at least one age-related transcriptomic change.

22. The reprogrammed cortical neuron or neuronal spheroids of claim 20, wherein the at least one transcription factor comprises NEUROD2, ASCL1 and MYT1L.

23. The reprogrammed cortical neuron or neuronal spheroids of claim 22, wherein the at least one transcription factor comprises NEUROD2 and MYT1L.
